(19) United States
(12) Patent Application Publication
(10) Pub. No.: US 2022/0160650 A1
(43) Pub. Date: May 26, 2022

(54) GEL, OINTMENT, AND FOAM FORMULATIONS OF TAPINAROF AND METHODS OF USE

(71) Applicant: Dermavant Sciences GmbH, Basel (CH)

(72) Inventors: Piyush JAIN, Durham, NC (US); Glenn TABOLT, Varina, NC (US); David Scott RUBENSTEIN, Chapel Hill, NC (US); Haripriya KALLURI, Morrisville, NC (US); Reinilda CATUBIG, Mulgrave (AU); Biljana ROUGHAN, Mulgrave (AU); Richard BUCHTA, Mulgrave (AU); Florencia Maria GUIDALI BONJOUR, London (GB); Brendan Philip BRADY, Guildford (GB); Laura Helen SUTCLIFFE, Guildford (GB)

(21) Appl. No.: 17/533,885

(22) Filed: Nov. 23, 2021

Related U.S. Application Data
(60) Provisional application No. 63/117,235, filed on Nov. 23, 2020.

(51) Int. Cl. A61K 31/05; A61K 9/06; A61K 47/10; A61K 9/12; A61K 9/00; A61K 47/12; A61K 47/26; A61K 47/18

(52) U.S. Cl. CPC A61K 31/05; A61K 9/06; A61K 47/10; A61K 47/183; A61K 9/0014; A61K 47/12; A61K 47/26; A61K 9/122

(57) ABSTRACT

Embodiments described herein relate to topical pharmaceutical compositions comprising tapinarof, wherein the topical pharmaceutical composition is formulated as an aqueous gel, an anhydrous gel, an ointment, or a foam. Embodiments also relate to methods of treating a dermatological condition or disorder in a patient by administering the present compositions to the skin of the patient.

### CROSS-REFERENCE TO RELATED APPLICATIONS

This application claims priority to and benefit of U.S. Provisional Application No. 63/117,235 filed Nov. 23, 2020, the entire content of which is hereby incorporated by reference.

### SUMMARY OF THE INVENTION

A challenge for the formulation chemist is to prepare a physically stable topical pharmaceutical composition where the active ingredient is also found to be chemically stable and the formulation provides the desired release profile for tapinarof and indication of interest. Such pharmaceutical compositions should: (i) not irritate the skin, (ii) be specifically adapted to deliver the active ingredient onto or into the skin so as to treat a particular dermatological condition or disorder, (iii) be cosmetically elegant to ensure that the patient complies with the prescribed treatment regimen, (iv) provide penetration of the active ingredient to the appropriate layers of the skin and engage the desired target, and (v) minimize systemic exposure while achieving local dermal/epidermal delivery.

One active ingredient of interest to be formulated in a physically and chemically stable topical composition is 3,5-Dihydroxy-4-isopropyl-trans-stilbene, which has the following formula:

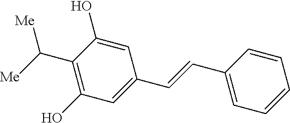 [SMILES: CC(C)c1c(O)cc(/C=C/c2ccccc2)cc1O]

This compound is also known as 5-[(E)-2-phenylethenyl]-2-(propan-2-yl)benzene-1,3-diol or 2-(1-Methyethyl)-5-[(1E)-2-phenylethenyl]-1,3-benzenediol, or tapinarof.

3,5-Dihydroxy-4-isopropyl-trans-stilbene is known to be sensitive to oxidation and photo degradation (see e.g., Gao et al., Journal of Polymer Research 2011 18: 1501-1508). Accordingly, there remains a need in the art for a topical composition comprising 3,5-Dihydroxy-4-isopropyl-trans-stilbene that is both chemically and physically stable, which delivers the active ingredient to the desired site of action in the epidermis and/or dermis, and which does not irritate the skin in use. The physical stability is demonstrated through visible appearance, maintenance of formulation consistency, consistent viscosity, and consistent pH. The chemical stability is demonstrated through the analysis of drug concentration and lack of degradation, as well as impurity analysis. Studies are performed as accelerated stability studies for 2 weeks at 40° C., 75% RH.

Embodiments described herein are directed to topical pharmaceutical aqueous gel compositions, anhydrous gel compositions, oleaginous gels, ointments wherein tapinarof is solubilized, ointments wherein tapinarof is suspended, silicone-based ointments, foams, PEG-based foams, cream-based foams, and hybrid emulsions and are not directed to creams or lotions formulated as oil-in-water emulsions, microemulsions, or nanoemulsions.

Embodiments described herein are directed to topical pharmaceutical aqueous gel compositions comprising: about 1% to about 4% tapinarof, or pharmaceutically acceptable salt thereof, about 10% to about 65% water, about 10% to about 50% diethylene glycol monoethyl ether (DEGEE), about 5% to about 65% of a glycol, about 2% to about 55% of a solvent, about 0.5% to about 5% of a gelling agent, and a neutralizing agent. In embodiments described herein, the topical pharmaceutical aqueous gel compositions have a pH of about 6 to about 6.5.

Embodiments described herein are directed to topical pharmaceutical anhydrous gel compositions comprising: about 1% to about 4% tapinarof, about 10% to about 70% of a solvent, about 10% to about 30% diethylene glycol monoethyl ether (DEGEE), about 15% to about 50% of a glycol, and about 0.5% to about 5% of a gelling agent.

Embodiments described herein are directed to topical pharmaceutical oleaginous gel compositions comprising: about 1% to about 4% tapinarof, about 15% of a glycol, about 35% to about 40% oil, about 35% to about 40% of an emollient, and about 10% of a gelling agent.

Embodiments described herein are directed to topical pharmaceutical ointment compositions comprising: about 1% to about 4% tapinarof, about 10% to about 70% of a low molecular weight PEG, about 50% to about 75% of a solvent, and about 25% to about 35% of a high molecular weight PEG.

Embodiments described herein are directed to topical pharmaceutical ointment compositions comprising: about 1% to about 4% tapinarof, about 5% to about 50% mineral oil, and about 49% to about 94% white petrolatum (white soft paraffin).

Embodiments described herein are directed to topical pharmaceutical ointment compositions comprising: about 1% to about 4% tapinarof, about 50% to about 60% petrolatum, and about 10% to about 40% of a silicone-based solvent. In certain embodiments, the topical pharmaceutical ointment composition further comprises about 10% isopropyl myristate (IPM), isopropyl palmitate (IPP), or combinations thereof.

Embodiments described herein are directed to topical pharmaceutical foam compositions comprising: about 1% to about 4% tapinarof, about 20% to about 80% of a solvent, about 0.5% to about 10% of an emulsifier, about 5% to about 15% of a thickener, and about 20% to about 50% of a propellant.

Embodiments described herein are directed to topical pharmaceutical foam compositions comprising about 1% to about 4% tapinarof, about 6% to about 15% diethylene glycol monoethyl ether (DEGEE), about 10% to about 40% PEG 400, about 4% to about 10% propylene glycol, about 0.5% to about 2% benzyl alcohol, about 3% to about 15% PEG 4000, about 0.5% to about 2% of an emulsifier, and about 20% to about 50% of a propellant.

Embodiments described herein are directed to topical pharmaceutical foam compositions comprising about 1% to about 4% tapinarof, about 30% to about 60% water, about 5% to about 20% propylene glycol, about 1% to about 15% of a solvent, about 1% to about 10% of an emulsifier, and about 20% of a propellant.

Embodiments described herein are directed to topical pharmaceutical foam compositions comprising a base composition of about 0.5% to about 2% tapinarof, about 85% to about 95% of a solvent, and about 3% to about 10% of an emulsifier, and about 5% to about 10% of the total weight of the base composition of a propellant.

Embodiments described herein are directed to topical pharmaceutical foam compositions comprising a base composition of about 1% to about 2% tapinarof, about 80% to about 90% of a solvent, and about 8% to about 15% of an emulsifier, and about 5% to about 10% of the total weight of the base composition of a propellant.

Embodiments described herein are directed to topical pharmaceutical emulsion compositions comprising: about 1% to about 4% tapinarof, about 15% glycerol, about 55% to about 60% water, about 10% isopropyl myristate, about 4% mixture of dimethicone and dimethiconol (such as dimethiconol blend 20), about 5% polydimethylsiloxane (such as TI-1050 fluid 350 Csp), and about 1.5% to about 10% of a gelling agent selected from Cyclopentasiloxane (and) Cyclohexasiloxane (and) Aluminum/Magnesium Hydroxide Stearate (such as Gilugel Sil 5), Sorbitan Sesquioleate (such as Span83), Hydroxyethyl Acrylate/Sodium Acryloyldimethyl Taurate Copolymer (such as Sepineo D.E.R.M.), or combination thereof.

Embodiments described herein are directed to topical pharmaceutical emulsion compositions comprising: about 1% to about 4% tapinarof, about 5% glycerol, about 20% diethylene glycol monoethyl ether (DEGEE) or propylene glycol, about 20% Mixture of Triceteareth-4 phosphate and Ethylene glycol palmitostearate and Diethylene glycol palmitostearate (such as Sedefos 75), about 5% Labrafil M 2125 CS (Linoleoyl polyoxyl-6 glycerides NF/Linoleoyl macrogol-6 glycerides EP), about 10% mineral oil, about 5% Compritol 888 (Glyceryl Behenate), and about 35% Lauroglycol 90 (Propylene glycol monolaurate (type II) EP/NF).

Embodiments described herein are directed to methods of treating a dermatological condition or disorder in a patient in need thereof, the method comprising administering to said patient a topical pharmaceutical composition as described herein.

Embodiments described herein are also directed to the use of a topical pharmaceutical composition described herein in the manufacture of a medicament for the treatment of dermatological condition or disorder in a patient.

### DESCRIPTION OF THE DRAWINGS

FIG. 1 provides images representing foamability and foam structure of formulations F1, F2, and F3.

FIG. 2 provides images representing foamability and foam structure of formulation F5.

### DETAILED DESCRIPTION

In addition to creating a physically and chemically stable pharmaceutical formulation, the present invention also provides for a pharmaceutical formulation which is non-irritating to the skin upon application and use, or is one which is less irritating than any previous formulations used in the development of the active ingredient to date. Another aspect of the invention is a formulation that not only has superior skin penetration and target engagement of the appropriate receptors, but also has significant non-systemic exposure of the active ingredient to the patient upon application and use.

The singular forms “a,” “an,” and “the” include plural referents unless the context clearly dictates otherwise. Thus, for example, reference to an “excipient” includes a single excipient as well as two or more of the same or different excipients, and the like.

The word “about” when immediately preceding a numerical value means a range of plus or minus 10% of that value, e.g., “about 50” means 45 to 55, “about 25,000” means 22,500 to 27,500, etc., unless the context of the disclosure indicates otherwise, or is inconsistent with such an interpretation. Furthermore, the phrases “less than about” a value or “greater than about” a value should be understood in view of the definition of the term “about” provided herein.

The terms “administer,” “administering” and “administration” as used herein refer to any method which in sound medical practice delivers the pharmaceutical composition to a patient in such a manner as to provide the desired therapeutic effect.

The transitional term “comprising,” which is synonymous with “including,” “containing,” or “characterized by,” is inclusive or open-ended and does not exclude additional, un-recited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed subject matter. In some embodiments or claims where the term comprising is used as the transition phrase, such embodiments can also be envisioned with replacement of the term “comprising” with the terms “consisting of” or “consisting essentially of.”

The phrase “pharmaceutically acceptable” and “dermatologically acceptable” is employed herein to refer to those agents of interest/compounds, salts, compositions, dosage forms, etc., which are within the scope of sound medical judgment suitable for use in contact with the tissues of human beings and/or other mammals without excessive toxicity, irritation, allergic response, or other problem or complication, commensurate with a reasonable benefit/risk ratio. In some aspects, pharmaceutically acceptable or dermatologically acceptable means approved by a regulatory agency of the federal or a state government, or listed in the U.S. Pharmacopeia or other generally recognized pharmacopeia for use in mammals (e.g., animals), and more particularly, in humans.

The term “pharmaceutically acceptable salt thereof” refers to salts that are safe and effective for topical use in the patient and possess the desired pharmaceutical activity. Such salts include salts formed when an acidic proton is replaced with a metal ion (e.g., alkali metal ion, alkaline earth metal ion, or aluminum ion).

The terms “patient” and “subject” are interchangeable and may be taken to mean any living organism which may be treated with compounds or compositions provided for herein. As such, the terms “patient” and “subject” may comprise, but is not limited to, any non-human mammal, primate or human. In some embodiments, the patient or subject is an adult, teen, child, or infant. In some embodiments, the patient or subject is a human.

The term “composition” as used herein refers to a combination or a mixture of two or more different ingredients, components, or substances; e.g., a combination of antioxidants.

The terms “treatment” or “treating” of a dermatological condition or disorder encompasses alleviation of at least one symptom thereof, a reduction in the severity thereof, or the delay, prevention or inhibition of the progression thereof. Treatment need not mean that the condition or disorder is totally cured. A useful pharmaceutical composition herein need only to reduce the severity of the condition or disorder, reduce the severity of symptoms associated therewith, provide improvement to a patient's quality of life, or delay, prevent or inhibit the onset of the condition or disorder. A treatment need not be effective in every member of a population, e.g., a population of patients with atopic dermatitis, to have clinical utility, as is recognized in the medical and pharmaceutical arts.

The phrase “therapeutically effective amount” or “effective amount” is used herein to refer to an amount of the active ingredient sufficient to have a therapeutic effect upon administration, e.g., that amount which will cause an improvement or change in the condition for which it is applied when applied to the affected area repeatedly over a period chime. Effective amounts will vary with the particular condition being treated, the severity of the condition, the duration of the treatment, the stage of advancement of the condition, the body surface area affected with the clinical condition, and the specific components of the composition. An effective amount of the active ingredient for treatment of a condition or disorder can be determined by standard clinical techniques. Appropriate amounts in any given instance will be readily apparent to those skilled in the art or capable of determination by routine experimentation. The compositions are generally applied in topical manner to the affected area, i.e., localized application to the skin region where the clinical abnormality is manifest.

Concentrations, amounts, solubilities, and other numerical data may be presented herein in a range format. It is to be understood that such range format is used merely for convenience and brevity and should be interpreted flexibly to include not only the numerical values explicitly recited as the limit of the range, but also to include all the individual numerical values or subranges encompassed within that range as if each numerical value and sub-range is explicitly recited. All numbers expressing quantities, percentages or proportions, and other numerical values used in the specification, are to be understood as being modified in all instances by the term “about.” For example, a concentration range of 0.1 to 5 ng/ml should be interpreted to include not only the explicitly recited concentration limits of 0.1 ng/ml and 5 ng/ml but also to include individual concentrations such as 0.2 ng/ml, 0.8 ng/ml, 1.0 ng/ml 2.2 ng/ml, 3.6 ng/ml, and sub-ranges such as 0.3-2.5 ng/ml, 1.8-3.2 ng/ml, etc. This interpretation should apply regardless of the breadth of the range or the characteristic being described. Any concentration range, percentage range or ratio range recited herein is to be understood to include concentrations, percentages or ratios of any integer within that range and fractions thereof, such as one tenth and one hundredth of an integer, unless otherwise indicated.

As used herein, “topical” administration of the pharmaceutical emulsion composition refers to application to and diffusion through the stratum corneum, including application to an affected area, lesions, and broken skin.

As used herein, in the in vitro skin penetration studies with ex-vivo human abdominal skin dermatomed at a thickness of 500 microns (+/−100 microns); the terms “epidermis” is the top/superficial layer and includes the stratum corneum and tissue or layers down to the basement membrane, obtained by heat separation procedure, and the term “dermis” is the underlying layer (after a washing/tape striping procedure).

As used herein, the term “skin penetration” refers to the diffusion of the 3,5-Dihydroxy-4-isopropyl-trans-stilbene or a pharmaceutically acceptable salt thereof through the stratum corneum and into the epidermis and/or dermis of the skin.

As used herein, “solubilize” means dissolved in a particular phase in an amount ≥50% w/w, or ≥60% w/w, or ≥70% w/w, or ≥80% w/w, or ≥90% w/w or ≥95% w/w, based on the percent by weight of the final composition prepared.

Reference to low molecular weight PEG refers to polyethylene glycol with a molecular weight of up to and including 600.

Reference to high molecular weight PEG refers to polyethylene glycol with a molecular weight of greater than 600 up to 8000.

Unless otherwise indicated, all percentages are based on the percent by weight of the final composition prepared, and all totals equal 100% by weight.

Other terms used herein are intended to be defined by their well-known meanings in the art.

While particular embodiments of the present invention are described, the skilled artisan will appreciate that various changes and modifications can be made without departing from the spirit and scope of the invention.

By hereby reserving the right to proviso out or exclude any individual members of any such group, including any sub-ranges or combinations of sub-ranges within the group, that can be claimed according to a range or in any similar manner, less than the full measure of this disclosure can be claimed for any reason. Throughout this disclosure, various patents, patent applications and publications are referenced. The disclosures of these patents, patent applications and publications in their entireties are incorporated into this disclosure by reference in order to more fully describe the state of the art as known to those skilled therein as of the date of this disclosure. This disclosure will govern in the instance that there is any inconsistency between the patents, patent applications and publications cited and this disclosure.

### Tapinarof (3,5-Dihydroxy-4-isopropyl-trans-stilbene)

Throughout this disclosure, use of tapinarof or DMVT-505 is intended to refer to 3,5-Dihydroxy-4-isopropyl-trans-stilbene or a pharmaceutically acceptable salt thereof. In an embodiment, the 3,5-Dihydroxy-4-isopropyl-trans-stilbene or a pharmaceutically acceptable salt thereof is present in the topical pharmaceutical composition in an amount from about 1% to about 4% by weight, such as from about 1.5% to about 3.5% by weight, or about 2% to about 3% by weight, based on the total weight of the composition. In another embodiment, the 3,5-Dihydroxy-4-isopropyl-trans-stilbene or a pharmaceutically acceptable salt thereof is present in an amount from about 1% to about 2% by weight, based on the total weight of the composition. In one embodiment, the 3,5-Dihydroxy-4-isopropyl-trans-stilbene or a pharmaceutically acceptable salt thereof is present in an amount of about 2% to about 3% by weight. In one embodiment, the 3,5-Dihydroxy-4-isopropyl-trans-stilbene or a pharmaceutically acceptable salt thereof is present in an amount of about 3% to about 4% by weight. In an embodiment, the 3,5-Dihydroxy-4-isopropyl-trans-stilbene or a pharmaceutically acceptable salt thereof is present in an amount of about 1%, 1.25%, 1.5%, 1.75%, 2%, 2.25%, 2.5%, 2.75%, 3%, 3.25%, 3.5%, 3.75%, or 4% by weight, based on the total weight of the composition.

Hybrid Emulsions

In certain embodiments, the topical pharmaceutical emulsion composition comprises about 1% tapinarof, about 5% glycerol, about 20% diethylene glycol monoethyl ether (DEGEE), about 20% mixture of triceteareth-4 phosphate and ethylene glycol palmitostearate and diethylene glycol palmitostearate (such as Sedefos 75), about 5% linoleoyl polyoxyl-6 glycerides NF/linoleoyl macrogol-6 glycerides EP (such as Labrafil M 2125 CS), about 10% mineral oil, 5% glyceryl behenate (such as Compritol 888), and about 35% propylene glycol monolaurate type II EP/NF (such as Lauroglycol 90).

In certain embodiments, the topical pharmaceutical emulsion composition comprises about 1% tapinarof, about 5% glycerol, about 20% propylene glycol, about 20% mixture of triceteareth-4 phosphate and ethylene glycol palmitostearate and diethylene glycol palmitostearate (such as Sedefos 75), about 5% linoleoyl polyoxyl-6 glycerides NF/linoleoyl macrogol-6 glycerides EP (such as Labrafil M 2125 CS), about 10% mineral oil, 5% glyceryl behenate (such as Compritol 888), and about 35% propylene glycol monolaurate type II EP/NF (such as Lauroglycol 90).

In certain embodiments, the topical pharmaceutical emulsion composition comprises about 1% tapinarof, about 10% isopropyl myristate, about 4% mixture of dimethicone and dimethiconol (such as Dimethiconol Blend 20), about 5% polydimethylsiloxane (such as TI-1050 fluid 350 cps), about 5% cyclopentasiloxane/cyclohexasiloxane/aluminum/magnesium hydroxide stearate (such as Gilugel SIL 5), about 2.5% sorbitan sesquioleate (such as Span 83), about 55% water, about 15% glycerol, about 1% phenoxyethanol, and about 1% sodium chloride.

In certain embodiments, the topical pharmaceutical emulsion composition comprises about 1% tapinarof, about 10% isopropyl myristate, about 4% mixture of dimethicone and dimethiconol (such as Dimethiconol Blend 20), about 5% polydimethylsiloxane (such as TI-1050 fluid 350 cps), about 2.5% sorbitan sesquioleate (such as Span 83), about 1.5% hydroxyethyl acrylate/sodium acryloyldimethyl taurate copolymer (such as Sepineo D.E.R.M.), about 60% water, about 15% glycerol, and about 1% phenoxyethanol.

Gels

Aqueous Gels/Hydrogels

Embodiments described herein are directed to topical pharmaceutical aqueous gel compositions comprising: about 1% to about 4% tapinarof, about 10% to about 65% water, about 10% to about 50% diethylene glycol monoethyl ether (DEGEE), about 5% to about 65% of a glycol, about 2% to about 55% of a solvent, about 0.5% to about 5% of a gelling agent, and a neutralizing agent. In embodiments described herein, the topical pharmaceutical aqueous gel compositions have a pH of about 6 to about 6.5.

In embodiments described herein, the topical pharmaceutical aqueous gel compositions contain solvent(s) which solubilize tapinarof. In certain embodiments, the solvent is selected from the group consisting of N-methyl-2-pyrrolidone (NMP), dimethyl sulfoxide (DMSO), dimethyl isosorbide (DMI), benzyl alcohol, phenoxyethanol, ethanol, cetostearyl alcohol, isopropanol, glycerol, medium chain triglycerides, D-panthenol, isostearic acid (such as Prisorine 3505), liquid lanolin (such as liquid medilan ultra), oleic acid, PEG 10 dimethicone, and combinations thereof.

In certain embodiments, the glycol is selected from the group consisting of low molecular weight PEG, PEG 200, PEG 300, PEG 400, propylene glycol, dipropylene glycol, hexylene glycol, butylene glycol, and combinations thereof.

In certain embodiments, the gelling agent is selected from the group consisting of a carbomer, an acrylate-based polymer, a cellulose-based polymer, a poloxamer, a high molecular weight PEG, a polyamide, a siligel, aluminum starch octenylsuccinate, and combinations thereof. The carbomer is selected from crosslinked polyacrylic acid (such as Carbopol® 980), carboxypolymethylene and carbomers (such as Carbopol® 974), crosslinked polyacrylic acid (such as Carbomer Ultrez 10), long chain alkyl acrylate with a lipophilic modification to its chemical backbone (such as Carbomer Ultrez 1342), or combinations thereof. The acrylate-based polymer is selected from crosslinked copolymer of acrylic acid and a hydrophobic C10-30 alkyl acrylate co-monomer (such as Pemulen TR-1 or Pemulen TR-2), Hydroxyethyl Acrylate/Sodium Acryloyldimethyl Taurate Copolymer (such as Sepineo D.E.R.M.), Acrylamide/Sodium Acryloyldimethyl Taurate Copolymer/Isohexadecane & Polysorbate 80 (such as Sepineo P 600), Polyacrylate Crosspolymer-6 (such as Sepineo PHD 100), or combinations thereof. The cellulose-based polymer is selected from hydroxypropylcellulose (HPC, such as HPC-HF, HPC-JF, HPC-MF), hydroxyethylcellulose (HEC, such as Natrosol 250 HHX), hydroxypropyl methylcellulose (HPMC, such as Benecel E4M pharm), or combinations thereof. The poloxamer is selected from Ethylene oxide/propylene oxide copolymer (such as Poloxamer 188), triblock copolymer consisting of a central hydrophobic block of polypropylene glycol flanked by two hydrophilic blocks of polyethylene glycol (PEG) (such as Poloxamer 407), or combinations thereof. The high molecular weight PEG is selected from PEG 1500, PEG 3350, PEG 4000, or combinations thereof. The polyamide is selected from Polyamide-3 (such as OleoCraft polyamide HP-31 or OleoCraft polyamide MP-30), Polyamide-8 (such as OleoCraft polyamide LP-20), or combinations thereof. The siligel is selected from xanthan gum, lecitihin, sclerotium gum, pullulan, or combinations thereof.

In certain embodiments, the neutralizing agent is used in an amount to adjust the pH of the composition to about 6 to about 6.5. In certain embodiments, the neutralizing agent is selected from the group consisting of sodium hydroxide, ammonium hydroxide, potassium hydroxide, arginine, aminomethyl propanol, tetrahydroxypropyl ethylenediamine, triethanolamine, tromethamine, PEG-15 cocamine, diisopropanolamine, triisopropanolamine, trolamine, and combinations thereof.

In certain embodiments, the topical pharmaceutical aqueous gel compositions further comprise an emulsifier selected from the group consisting of polysorbate 80, polyoxyethylene (2) stearyl ether (such as Brij S2), polyoxyethylene (20) stearyl ether (such as Brij S20), Polawax NF, glyceryl monostearate, and combinations thereof.

In certain embodiments, the topical pharmaceutical aqueous gel composition comprises about 2.4% tapinarof, about 27.6% water, about 21% diethylene glycol monoethyl ether (DEGEE), about 10% PEG400, about 23% propylene glycol, about 1% phenoxyethanol, and about 1% crosslinked polyacrylic acid (such as Carboppol 980). In certain embodiments, the topical pharmaceutical aqueous gel composition has a pH of about 6 to about 6.5.

In certain embodiments, the topical pharmaceutical aqueous gel composition comprises about 2.4% tapinarof, about 27.6% water, about 21% diethylene glycol monoethyl ether (DEGEE), about 10% PEG400, about 23% propylene glycol, about 1% phenoxyethanol, and about 1% crosslinked copolymer acrylic acid and a hydrophobic C10-30 alkyl acrylate co-monomer (such as Pemulen TR-1 or Pemulen TR-2). In certain embodiments, the topical pharmaceutical aqueous gel composition has a pH of about 6 to about 6.5.

In certain embodiments, the topical pharmaceutical aqueous gel composition comprises about 2.4% tapinarof, about 41.1% water, about 20% diethylene glycol monoethyl ether (DEGEE), about 10% PEG400, about 23% propylene glycol, about 2% benzyl alcohol, and about 1.5% hydroxyethyl acrylate/sodium acryloyldimethyl taurate copolymer (such as Sepineo D.E.R.M.).

In certain embodiments, the topical pharmaceutical aqueous gel composition comprises about 1% tapinarof, about 25% water, about 20% diethylene glycol monoethyl ether (DEGEE), about 10% PEG400, about 23% N-methyl-2-pyrrolidone (NMP), about 1% phenoxyethanol, and about 1% crosslinked polyacrylic acid (such as Carboppol 980). In certain embodiments, the topical pharmaceutical aqueous gel composition has a pH of about 6 to about 6.5.

In certain embodiments, the topical pharmaceutical aqueous gel composition comprises about 1.8% tapinarof, about 25% water, about 20% diethylene glycol monoethyl ether (DEGEE), about 10% PEG400, about 23% dimethyl sulfoxide (DMSO), about 1% phenoxyethanol, and about 1% crosslinked polyacrylic acid (such as Carboppol 980). In certain embodiments, the topical pharmaceutical aqueous gel composition has a pH of about 6 to about 6.5.

In certain embodiments, the topical pharmaceutical aqueous gel composition comprises about 4% tapinarof, about 5% water, about 20% diethylene glycol monoethyl ether (DEGEE), about 40% PEG400, about 23% propylene glycol, about 2% benzyl alcohol, and about 1% crosslinked polyacrylic acid (such as Carboppol 980). In certain embodiments, the topical pharmaceutical aqueous gel composition has a pH of about 6 to about 6.5.

In certain embodiments, the topical pharmaceutical aqueous gel composition comprises about 1% tapinarof, about 30% water, about 20% diethylene glycol monoethyl ether (DEGEE), about 10% PEG400, about 23% dimethyl isosorbide (DMI), about 1% phenoxyethanol, and about 1% crosslinked polyacrylic acid (such as Carboppol 980). In certain embodiments, the topical pharmaceutical aqueous gel composition has a pH of about 6 to about 6.5.

In certain embodiments, the topical pharmaceutical aqueous gel composition comprises about 1% tapinarof, about 30% water, about 20% diethylene glycol monoethyl ether (DEGEE), about 23% PEG400, about 10% dimethyl sulfoxide (DMSO), about 1% phenoxyethanol, and about 1% crosslinked polyacrylic acid (such as Carboppol 980). In certain embodiments, the topical pharmaceutical aqueous gel composition has a pH of about 6 to about 6.5.

In certain embodiments, the topical pharmaceutical aqueous gel composition comprises about 1% tapinarof, about 20% water, about 12.8% diethylene glycol monoethyl ether (DEGEE), about 6.4% PEG400, about 50% dimethyl sulfoxide (DMSO), about 1% phenoxyethanol, and about 1% crosslinked polyacrylic acid (such as Carboppol 980). In certain embodiments, the topical pharmaceutical aqueous gel composition has a pH of about 6 to about 6.5.

In certain embodiments, the topical pharmaceutical aqueous gel composition comprises about 2% tapinarof, about 40% water, about 49.1% diethylene glycol monoethyl ether (DEGEE), about 1% phenoxyethanol, and about 1% crosslinked polyacrylic acid (such as Carboppol 980). In certain embodiments, the topical pharmaceutical aqueous gel composition has a pH of about 6 to about 6.5.

In certain embodiments, the topical pharmaceutical aqueous gel composition comprises about 4% tapinarof, about 30% water, about 20% diethylene glycol monoethyl ether (DEGEE), about 10% PEG400, about 23% dipropylene glycol, about 2% benzyl alcohol, and about 1% crosslinked polyacrylic acid (such as Carboppol 980). In certain embodiments, the topical pharmaceutical aqueous gel composition has a pH of about 6 to about 6.5.

In certain embodiments, the topical pharmaceutical aqueous gel composition comprises about 0.1% tapinarof, about 58.9% water, about 15% diethylene glycol monoethyl ether (DEGEE), about 10% PEG400, about 13% propylene glycol, about 2% benzyl alcohol, and about 1% Natrosol 250 HHX (HEC). In certain embodiments, the topical pharmaceutical aqueous gel composition has a pH of about 6 to about 6.5.

In certain embodiments, the topical pharmaceutical aqueous gel composition comprises about 2.4% tapinarof, about 41.6% water, about 20% diethylene glycol monoethyl ether (DEGEE), about 10% PEG400, about 23% propylene glycol, about 2% benzyl alcohol, and about 1% Natrosol 250 HHX (HEC). In certain embodiments, the topical pharmaceutical aqueous gel composition has a pH of about 6 to about 6.5.

In certain embodiments, the topical pharmaceutical aqueous gel composition comprises about 2.4% tapinarof, about 10% diethylene glycol monoethyl ether (DEGEE), about 62.9% water, about 1% phenoxyethanol, about 5% glycerol, about 4% acrylamide/sodium acryloyldimethyl taurate copolymer/isohexadecane/polysorbate 80 (such as Sepineo P600), about 0.1% BHT, about 10% medium chain triglycerides, about 1% dimethicone, and about 5% isopropyl myristate.

In certain embodiments, the topical pharmaceutical aqueous gel composition comprises about 4% tapinarof, about 45.45% PEG400, about 5% propylene glycol, about 10% ethanol, about 15% diethylene glycol monoethyl ether (DEGEE), about 10% water, about 1% phenoxyethanol, about 1% triblock copolymer consisting of a central hydrophobic block of polypropylene glycol flanked by two hydrophilic blocks of polyethylene glycol (such as Poloxamer 407), about 1% crosslinked polyacrylic acid (such as Carbomer Ultrez 10), about 0.05% propyl gallate, and about 7.5% cyclomethicone. In certain embodiments, the topical pharmaceutical aqueous gel composition has a pH of about 6 to about 6.5.

In certain embodiments, the topical pharmaceutical aqueous gel composition comprises about 1% tapinarof, about 28.45% PEG400, about 5% propylene glycol, about 10% ethanol, about 15% diethylene glycol monoethyl ether (DEGEE), about 30% water, about 1% phenoxyethanol, about 1% triblock copolymer consisting of a central hydrophobic block of polypropylene glycol flanked by two hydrophilic blocks of polyethylene glycol (such as Poloxamer 407), about 1% crosslinked polyacrylic acid (such as Carbomer Ultrez 10), about 0.05% propyl gallate, and about 7.5% cyclomethicone. In certain embodiments, the topical pharmaceutical aqueous gel composition has a pH of about 6 to about 6.5.

Anhydrous Gels

Embodiments described herein are directed to topical pharmaceutical anhydrous gel compositions comprising: about 1% to about 4% tapinarof, about 10% to about 70% of a solvent, about 10% to about 30% diethylene glycol monoethyl ether (DEGEE), about 15% to about 50% of a glycol, and about 0.5% to about 5% of a gelling agent.

In embodiments described herein, the topical pharmaceutical anhydrous gel compositions contain solvent(s) which solubilize tapinarof. In certain embodiments, the solvent is selected from the group consisting of N-methyl-2-pyrrolidone (NMP), dimethyl sulfoxide (DMSO), dimethyl isosorbide (DMI), benzyl alcohol, phenoxyethanol, ethanol, cetostearyl alcohol, isopropanol, glycerol, medium chain triglycerides, D-panthenol, isostearic acid (such as Prisorine 3505), liquid lanolin (such as liquid medilan ultra), oleic acid, PEG 10 dimethicone, and combinations thereof.

In certain embodiments, the glycol is selected from the group consisting of low molecular weight PEG, PEG 200, PEG 300, PEG 400, propylene glycol, dipropylene glycol, hexylene glycol, butylene glycol, and combinations thereof.

In certain embodiments, the gelling agent is selected from the group consisting of a carbomer, an acrylate-based polymer, a cellulose-based polymer, a poloxamer, a high molecular weight PEG, a polyamide, a siligel, aluminum starch octenylsuccinate, and combinations thereof. The carbomer is selected from crosslinked polyacrylic acid (such as Carbopol® 980), carboxypolymethylene and carbomers (such as Carbopol® 974), crosslinked polyacrylic acid (such as Carbomer Ultrez 10), long chain alkyl acrylate with a lipophilic modification to its chemical backbone (such as Carbomer Ultrez 1342), or combinations thereof. The acrylate-based polymer is selected from crosslinked copolymer of acrylic acid and a hydrophobic C10-30 alkyl acrylate co-monomer (such as Pemulen TR-1 or Pemulen TR-2), Hydroxyethyl Acrylate/Sodium Acryloyldimethyl Taurate Copolymer (such as Sepineo D.E.R.M.), Acrylamide/Sodium Acryloyldimethyl Taurate Copolymer/Isohexadecane & Polysorbate 80 (such as Sepineo P 600), Polyacrylate Crosspolymer-6 (such as Sepineo PHD 100), or combinations thereof. The cellulose-based polymer is selected from hydroxypropylcellulose (HPC, such as HPC-HF, HPC-JF, HPC-MF), hydroxyethylcellulose (HEC, such as Natrosol 250 HHX), hydroxypropyl methylcellulose (HPMC, such as Benecel E4M pharm), or combinations thereof. The poloxamer is selected from Ethylene oxide/propylene oxide copolymer (such as Poloxamer 188), triblock copolymer consisting of a central hydrophobic block of polypropylene glycol flanked by two hydrophilic blocks of polyethylene glycol (PEG) (such as Poloxamer 407), or combinations thereof. The high molecular weight PEG is selected from PEG 1500, PEG 3350, PEG 4000, or combinations thereof. The polyamide is selected from Polyamide-3 (such as OleoCraft polyamide HP-31 or OleoCraft polyamide MP-30), Polyamide-8 (such as OleoCraft polyamide LP-20), or combinations thereof. The siligel is selected from xanthan gum, lecitihin, sclerotium gum, pullulan, or combinations thereof.

In certain embodiments, the topical pharmaceutical anhydrous gel compositions further comprise an emulsifier selected from the group consisting of polysorbate 80, polyoxyethylene (2) stearyl ether (such as Brij S2), polyoxyethylene (20) stearyl ether (such as Brij S20), Polawax NF, glyceryl monostearate, and combinations thereof.

In certain embodiments, the topical pharmaceutical anhydrous gel composition comprises about 1% tapinarof, about 20% diethylene glycol monoethyl ether (DEGEE), about 20% glycerol, about 33% PEG200, about 15% propylene glycol, about 10% ethanol, and about 1% HPC-HF.

In certain embodiments, the topical pharmaceutical anhydrous gel composition comprises about 4% tapinarof, about 20% diethylene glycol monoethyl ether (DEGEE), about 20% glycerol, about 28% PEG200, about 15% propylene glycol, about 10% ethanol, and about 1% HPC-HF.

In certain embodiments, the topical pharmaceutical anhydrous gel composition comprises about 1% tapinarof, about 20% diethylene glycol monoethyl ether (DEGEE), about 20% glycerol, about 32% PEG200, about 15% propylene glycol, about 10% ethanol, and about 2% HPC-MF.

In certain embodiments, the topical pharmaceutical anhydrous gel composition comprises about 1% tapinarof, about 25% diethylene glycol monoethyl ether (DEGEE), about 20% glycerol, about 42% PEG400, about 10% N-methyl-2-pyrrolidine (NMP), and about 2% HPC-MF.

In certain embodiments, the topical pharmaceutical anhydrous gel composition comprises about 1% tapinarof, about 25% diethylene glycol monoethyl ether (DEGEE), about 20% glycerol, about 42% PEG400, about 10% dimethyl sulfoxide (DMSO), and about 2% HPC-MF.

In certain embodiments, the topical pharmaceutical anhydrous gel composition comprises about 1% tapinarof, about 20.84% diethylene glycol monoethyl ether (DEGEE), about 40.68% glycerol, about 10.42% PEG200, about 15.63% propylene glycol, about 10.42% ethanol, and about 1% carboxymethylene and carbomers (such as Carbopol 974). In certain embodiments, the topical pharmaceutical aqueous gel composition has a pH of about 6 to about 6.5.

In certain embodiments, the topical pharmaceutical anhydrous gel composition comprises about 1% tapinarof, about 13.63% diethylene glycol monoethyl ether (DEGEE), about 10.91% glycerol, about 23.45% PEG400, about 50% dimethyl sulfoxide (DMSO), and about 2% HPC-MF.

In certain embodiments, the topical pharmaceutical anhydrous gel composition comprises about 1% tapinarof, about 20% diethylene glycol monoethyl ether (DEGEE), about 20% glycerol, about 32% PEG200, about 15% hexylene glycol, about 10% ethanol, and about 2% HPC-MF.

In certain embodiments, the topical pharmaceutical anhydrous gel composition comprises about 1% tapinarof, about 20% diethylene glycol monoethyl ether (DEGEE), about 20% glycerol, about 32% PEG400, about 15% propylene glycol, about 10% ethanol, and about 2% HPC-MF.

In certain embodiments, the topical pharmaceutical anhydrous gel composition comprises about 1% tapinarof, about 30.5% glycerol, about 20% diethylene glycol monoethyl ether (DEGEE), about 31.5% propylene glycol, about 0.05% BHT, about 1.5% hydroxyethyl acrylate/sodium acryloyldimethyl taurate copolymer (such as Sepineo D.E.R.M.), and about 15.45% cyclomethicone 5NF.

Oleaginous Gel

Embodiments described herein are directed to topical pharmaceutical oleaginous gel compositions comprising: about 1% to about 4% tapinarof, about 15% of a glycol, about 35% to about 40% oil, about 35% to about 40% of an emollient, and about 10% of a gelling agent.

In certain embodiments, the glycol is selected from the group consisting of PPG (polypropylene glycol)-15 stearyl ether, low molecular weight PEG, PEG 200, PEG 300, PEG 400, propylene glycol, dipropylene glycol, hexylene glycol, butylene glycol, and combinations thereof.

In certain embodiments, the oil is selected from the group consisting of oils and fats include fatty acids, esters, esters of glycerin, fatty alcohols, waxes, sterols, unsaponifiables, siloxanes, silanes, lanolin, hydrocarbons, essential oils, vegetable oils, mineral oils, castor oil, animal oils, edible oils, and combinations thereof.

In an embodiment, the fatty acids include, but are not limited to, isostearic acid, oleic acid, stearic acid, linoleic acid, linolenic acid, myristic acid, palmitic acid, ricinoleic acid, arachidic acid, and combinations thereof.

In an embodiment, the esters include, but are not limited to, coco-caprylate/caprate, diethyl sebacate, diisopropyl adipate, diisopropyl dilinoleate, ethyl oleate, ethylhexyl hydroxystearate, glycol distearate, glycol stearate, hydroxyoctacosanyl hydroxystearate, isopropyl isostearate (Crodamol IPIS), isopropyl myristate, isopropyl palmitate, isopropyl stearate, methyl glucose sesquistearate, methyl laurate, methyl salicylate, methyl stearate, myristyl lactate, octyl salicylate, oleyl oleate, PPG-20 methyl glucose ether distearate, propylene glycol diacetate, propylene glycol dicaprylate, propylene glycol monolaurate, propylene glycol monopalmitostearate, propylene glycol ricinoleate, triacetin, sucrose distearate, cocoyl caprylocaprate (Kollicream 3C), isostearyl isostearate (Crodamol ISIS), octyldodecanol (Kollicream OD), and combinations thereof.

In an embodiment, the esters of glycerin include, but are not limited to, caprylic/capric glycerides, caprylic/capric triglyceride, caprylic/capric/succinic triglyceride, capryl glucoside, cetearyl glucoside, cocoglycerides, decyl glucoside, lauryl glucoside, glyceryl citrate, glyceryl isostearate, glyceryl laurate, glyceryl monostearate, glyceryl oleate, glyceryl palmitate, glyceryl ricinoleate, glyceryl stearate, mono and diglyceride, PEG-12 glyceryl laurate, PEG-120 glyceryl stearate, polyglyceryl-3 oleate, polyoxyl glyceryl stearate, tallow glycerides, medium chain triglycerides (MCT), and combinations thereof. In one embodiment, the medium chain triglyceride carbon length is from C₆ to C₁₂. In another embodiment, the medium chain triglyceride carbon length is from C6 to C14.

In an embodiment, the fatty alcohols include, but are not limited to, caprylic alcohol, decyl alcohol, lauryl alcohol, myristyl alcohol, behenyl alcohol, lanolin alcohol, arachidyl alcohol, oleyl alcohol (Kollicream OA), palm alcohol, isocetyl alcohol, cetyl alcohol, stearyl alcohol, and combinations thereof.

In an embodiment, the waxes include, but are not limited to, beeswax, camauba wax, dimethicone PEG-1 beeswax, dimethiconol beeswax, lanolin wax, microcrystalline wax, white wax, candelilla wax, paraffin wax, emulsifying wax, PEG-8 beeswax, yellow wax, cetyl esters wax, shellac wax, synthetic beeswax, and combinations thereof.

In an embodiment, the sterols include, but are not limited to, Brassica campestris sterols, C₁₀-C₃₀ cholesterol/lanosterol esters, canola sterols, cholesterol, lanolin cholesterols, Glycine soja sterols, PEG-20 phytosterol, phytosterols, and combinations thereof.

In an embodiment, the siloxanes and silanes include, but are not limited to, dimethicone, cyclomethicone, simethicone, phenyl dimethicone, cyclopentasiloxane, cyclotetrasiloxane, dimethyl siloxane, dimethicone cross polymer, and combinations thereof.

In an embodiment, the hydrocarbons include, but are not limited to, dodecane, petrolatum, squalane, squalene, paraffin, and combinations thereof.

In an embodiment, the essential oils include, but are not limited to, primrose oil, rose oil, eucalyptus oil, borage oil, bergamot of 1, chamomile oil, citronella oil, lavender oil, peppermint oil, pine oil, pine needle oil, spearmint oil, tea tree oil, wintergreen oil, jajoba oil, and combinations thereof.

In an embodiment, the vegetable oils include, but are not limited to, almond oil, aniseed oil, canola oil, castor oil, coconut oil, corn oil, avocado oil, cottonseed oil, olive oil, palm kernel oil, peanut oil, sunflower oil, safflower oil, soybean oil, sesame oil, walnut oil, and combinations thereof.

In an embodiment, the edible oils include, but are not limited to, cinnamon oil, clove oil, lemon oil and peppermint oil, and combinations thereof.

In certain embodiments, the emollient is selected from the group consisting of isopropyl myristate (IPM), octyldodecanol (such as Kollicream® OD), myristyl lactate, and combinations thereof.

In certain embodiments, the gelling agent is selected from the group consisting of a carbomer, an acrylate-based polymer, a cellulose-based polymer, a poloxamer, a high molecular weight PEG, a polyamide, a siligel, aluminum starch octenylsuccinate, and combinations thereof. The carbomer is selected from crosslinked polyacrylic acid (such as Carbopol® 980), carboxypolymethylene and carbomers (such as Carbopol® 974), crosslinked polyacrylic acid (such as Carbomer Ultrez 10), long chain alkyl acrylate with a lipophilic modification to its chemical backbone (such as Carbomer Ultrez 1342), or combinations thereof. The acrylate-based polymer is selected from crosslinked copolymer of acrylic acid and a hydrophobic C10-30 alkyl acrylate co-monomer (such as Pemulen TR-1 or Pemulen TR-2), Hydroxyethyl Acrylate/Sodium Acryloyldimethyl Taurate Copolymer (such as Sepineo D.E.R.M.), Acrylamide/Sodium Acryloyldimethyl Taurate Copolymer/Isohexadecane & Polysorbate 80 (such as Sepineo P 600), Polyacrylate Crosspolymer-6 (such as Sepineo PHD 100), or combinations thereof. The cellulose-based polymer is selected from hydroxypropylcellulose (HPC, such as HPC-HF, HPC-JF, HPC-MF), hydroxyethylcellulose (HEC, such as Natrosol 250 HHX), hydroxypropyl methylcellulose (HPMC, such as Benecel E4M pharm), or combinations thereof. The poloxamer is selected from Ethylene oxide/propylene oxide copolymer (such as Poloxamer 188), triblock copolymer consisting of a central hydrophobic block of polypropylene glycol flanked by two hydrophilic blocks of polyethylene glycol (PEG) (such as Poloxamer 407), or combinations thereof. The high molecular weight PEG is selected from PEG 1500, PEG 3350, PEG 4000, or combinations thereof. The polyamide is selected from Polyamide-3 (such as OleoCraft polyamide HP-31 or OleoCraft polyamide MP-30), Polyamide-8 (such as OleoCraft polyamide LP-20), or combinations thereof. The siligel is selected from xanthan gum, lecitihin, sclerotium gum, pullulan, or combinations thereof.

In certain embodiments, the topical pharmaceutical oleaginous gel composition comprises about 1% tapinarof, about 15% PPG-15 stearyl ether, about 24% mineral oil, about 15% castor oil, about 15% isopropyl myristate, about 15% octyldodecanol (Kollicream OD), about 10% myristyl lactate, and about 10% polyamide-8 (such as OleoCraft polyamide LP-20).

Ointments

Ointments Wherein Tapinarof is Solubilized

Embodiments described herein are directed to topical pharmaceutical ointment compositions comprising: about 1% to about 4% tapinarof, about 10% to about 70% of a low molecular weight PEG, about 50% to about 75% of a solvent, and about 25% to about 35% of a high molecular weight PEG.

In certain embodiments, the low molecular weight PEG is selected from the group consisting of PEG 200, PEG 300, PEG 400, PEG 500, PEG 600, and combinations thereof.

In embodiments described herein, the topical pharmaceutical ointment compositions contain solvent(s) which solubilize tapinarof. In certain embodiments, the solvent is selected from the group consisting of a glycol, diethylene glycol monoethyl ether (DEGEE), an alcohol, water, glycerol, dimethyl sulfoxide (DMSO), N-methyl-2-pyrrolidone (NMP), dimethyl isosorbide (DMI), medium chain triglycerides, D-panthenol, isostearic acid (such as Prisorine 3505), liquid lanolin (such as liquid medilan ultra), oleic acid, PEG 10 dimethicone, and combinations thereof. In certain embodiments, the glycol is selected from the group consisting of propylene glycol, hexylene glycol, dipropylene glycol, butylene glycol, and combinations thereof. In certain embodiments, the alcohol is selected from the group consisting of ethanol, isopropanol, benzyl alcohol, phenoxyethanol, cetostearyl alcohol, and combinations thereof.

In certain embodiments, the glycols are present in an amount of about 10% to about 40%.

In certain embodiments, the diethylene glycol monoethyl ether (DEGEE) is present in an amount of about 10% to about 40%.

In certain embodiments, the alcohol is present in an amount of about 1% to about 10%.

In certain embodiments, the water is present in an amount of about 1% to about 25%.

In certain embodiments, the glycerol is present in an amount of about 1% to about 25%.

In certain embodiments, the dimethyl sulfoxide (DMSO) is present in an amount of about 1% to about 50%.

In certain embodiments, the high molecular weight PEG is selected from the group consisting of PEG 1500, PEG 3350, PEG 4000, and combinations thereof.

In certain embodiments, the topical pharmaceutical ointment composition further comprises about 10% of a silicone-based solvent. In certain embodiments, the silicone-based solvent is selected from cyclomethicone D5, cyclomethicone D56, dimethicone CST 100, dimethicone CST 1000, dimethiconol 40, mixture of dimethicone and dimethiconol (such as dimethiconol blend 20), crosspolymer of Cyclopentasiloxane and Dimethicone (such as elastomer 10), mixture of Disiloxane (0.65 cSt) and Trisiloxane (1 cSt) (such as silicone fluid Q7-9180), mixture of Stearoxytrimethylsilane and stearyl alcohol (such as silky wax 10), mixture of Dimethicone and Dimethiconol (such as TI-3011 gum blend), Silicone elastomer blended with a low viscosity (5 cSt) dimethicone (such as TI-3021 silicone elastomer blend), or combinations thereof.

In certain embodiments, the topical pharmaceutical ointment composition comprises about 1% tapinarof, about 68.9% PEG400, about 5% ethanol, about 0.1% BHT, and about 25% PEG3350.

In certain embodiments, the topical pharmaceutical ointment composition comprises about 4% tapinarof, about 64.9% PEG400, about 5% ethanol, about 0.1% BHT, and about 25% PEG3350.

In certain embodiments, the topical pharmaceutical ointment composition comprises about 1% tapinarof, about 48.9% PEG400, about 25% glycerol, about 0.1% BHT, and about 25% PEG3350.

In certain embodiments, the topical pharmaceutical ointment composition comprises about 1% tapinarof, about 43.9% PEG400, about 15% propylene glycol, about 10% diethylene glycol monoethyl ether (DEGEE), about 0.1% BHT, and about 30% PEG3350.

In certain embodiments, the topical pharmaceutical ointment composition comprises about 1% tapinarof, about 33.95% PEG400, about 20% water, about 20% glycerol, about 0.05% propyl gallate, and about 25% PEG3350.

In certain embodiments, the topical pharmaceutical ointment composition comprises about 1% tapinarof, about 33.9% PEG400, about 40% diethylene glycol monoethyl ether (DEGEE), about 0.1% BHT, and about 25% PEG3350.

In certain embodiments, the topical pharmaceutical ointment composition comprises about 1% tapinarof, about 53.9% PEG400, about 10% dimethicone CST 100, about 0.1% BHT, and about 35% PEG3350.

In certain embodiments, the topical pharmaceutical ointment composition comprises about 1% tapinarof, about 68.9% PEG400, about 5% ethanol, about 0.1% BHT, and about 25% PEG1500.

In certain embodiments, the topical pharmaceutical ointment composition comprises about 1% tapinarof, about 68.9% PEG400, about 5% ethanol, about 0.1% BHT, and about 25% PEG4000.

In certain embodiments, the topical pharmaceutical ointment composition comprises about 1% tapinarof, about 13.9% PEG400, about 50% dimethyl sulfoxide (DMSO), about 0.1% BHT, and about 35% PEG3350.

In certain embodiments, the topical pharmaceutical ointment composition comprises about 1% tapinarof, about 5% mineral oil, and about 94% white petrolatum.

Ointments Wherein Tapinarof is Suspended (Oleaginous Ointments)

Embodiments described herein are directed to topical pharmaceutical ointment compositions comprising: about 1% to about 4% tapinarof, about 5% to about 50% mineral oil, and about 49% to about 94% white petrolatum (white soft paraffin).

Silicone-Based Ointments

Embodiments described herein are directed to topical pharmaceutical ointment compositions comprising: about 1% to about 4% tapinarof, about 50% to about 60% petrolatum, and about 10% to about 40% of a silicone-based solvent. In certain embodiments, the topical pharmaceutical ointment composition further comprises about 10% isopropyl myristate (IPM), isopropyl palmitate (IPP), or combinations thereof.

In certain embodiments, the silicone-based solvent is selected from the group consisting of cyclomethicone D5, cyclomethicone D56, dimethicone CST 100, dimethicone CST 1000, dimethiconol 40, mixture of dimethicone and dimethiconol (such as dimethiconol blend 20), crosspolymer of Cyclopentasiloxane and Dimethicone (such as elastomer 10), mixture of Disiloxane (0.65 cSt) and Trisiloxane (1 cSt) (such as silicone fluid Q7-9180), mixture of Stearoxytrimethylsilane and stearyl alcohol (such as silky wax 10), mixture of Dimethicone and Dimethiconol (such as TI-3011 gum blend), Silicone elastomer blended with a low viscosity (5 cSt) dimethicone (such as TI-3021 silicone elastomer blend), or combinations thereof.

In certain embodiments, the topical pharmaceutical ointment composition comprises about 1% tapinarof, about 59% petrolatum, about 20% silicone elastomer blend (such as Dow Corning TI-3021), about 10% dimethicone 100 cps, and about 10% IPP.

In certain embodiments, the topical pharmaceutical ointment composition comprises about 1% tapinarof, about 59% petrolatum, about 20% silicone elastomer blend (such as Dow Corning TI-3021), about 10% cyclomethicone SNF, and about 10% IPP.

In certain embodiments, the topical pharmaceutical ointment composition comprises about 1% tapinarof, about 59% petrolatum, about 20% elastomer 10, about 10% cyclomethicone SNF, and about 10% IPP.

Foams

Embodiments described herein are directed to topical pharmaceutical foam compositions comprising: about 1% to about 4% tapinarof, about 20% to about 80% of a solvent, about 0.5% to about 10% of an emulsifier, about 5% to about 15% of a thickener, and about 20% to about 50% of a propellant.

Embodiments described herein are directed to topical pharmaceutical foam compositions comprising a base composition of about 1% to about 2% tapinarof, about 80% to about 90% of a solvent, and about 8% to about 15% of an emulsifier, and about 5% to about 10% of the total weight of the base composition of a propellant.

In embodiments described herein, the topical pharmaceutical foam compositions contain solvent(s) which solubilize tapinarof. In certain embodiments, the solvents are selected from the group consisting of solvent is selected from the group consisting of a glycol, diethylene glycol monoethyl ether (DEGEE, also known as Transcutol P), an alcohol, water, glycerol, dimethyl sulfoxide (DMSO), N-methyl-2-pyrrolidone (NMP), dimethyl isosorbide (DMI), medium chain triglycerides (such as Migyol 812N), D-panthenol, isostearic acid (such as Prisorine 3505), liquid lanolin (such as liquid medilan ultra), oleic acid, PEG 10 dimethicone, and combinations thereof. In certain embodiments, the glycol is selected from the group consisting of low molecular weight PEG, PEG 200, PEG 300, PEG 400, propylene glycol, dipropylene glycol, hexylene glycol, butylene glycol, and combinations thereof. In certain embodiments, the alcohol is selected from the group consisting of ethanol, isopropanol, benzyl alcohol, phenoxyethanol, cetostearyl alcohol, cetyl alcohol, and combinations thereof.

In certain embodiments, the glycols are present in an amount of about 1% to about 15%.

In certain embodiments, the diethylene glycol monoethyl ether (DEGEE) is present in an amount of about 1% to about 15%.

In certain embodiments, the low molecular weight PEG is present in an amount of about 10% to about 40%.

In certain embodiments, the water is present in an amount of about 1% to about 52%. In certain embodiments, the water is present in an amount of about 65% to about 75%.

In certain embodiments, the glycerol is present in an amount of about 1% to about 30%.

In embodiments described herein, the topical pharmaceutical foam compositions contain emulsifiers used to stabilize the foam. In certain embodiments, the emulsifiers are selected from the group consisting of steareth 2, steareth 20, polysorbate 80, polyoxyethylene (2) stearyl ether (such as Brij S2), polyoxyethylene (20) stearyl ether (such as Brij S20), Polawax NF, glyceryl monostearate, Kolliwax CSA50, Kolliphor CS20, cocoyl caprylocaprate, isopropyl palmitate, and combination thereof.

In embodiments described herein, the topical pharmaceutical foam compositions contain thickeners used to stabilize the foam. In certain embodiments, the thickeners are selected from the group consisting of a carbomer, an acrylate-based polymer, a cellulose-based polymer, a poloxamer, a high molecular weight PEG, a polyamide, a siligel, aluminum starch octenylsuccinate, and combinations thereof. The carbomer is selected from crosslinked polyacrylic acid (such as Carbopol® 980), carboxypolymethylene and carbomers (such as Carbopol® 974), crosslinked polyacrylic acid (such as Carbomer Ultrez 10), long chain alkyl acrylate with a lipophilic modification to its chemical backbone (such as Carbomer Ultrez 1342), or combinations thereof. The acrylate-based polymer is selected from crosslinked copolymer of acrylic acid and a hydrophobic C10-30 alkyl acrylate co-monomer (such as Pemulen TR-1 or Pemulen TR-2), Hydroxyethyl Acrylate/Sodium Acryloyldimethyl Taurate Copolymer (such as Sepineo D.E.R.M.), Acrylamide/Sodium Acryloyldimethyl Taurate Copolymer/Isohexadecane & Polysorbate 80 (such as Sepineo P 600), Polyacrylate Crosspolymer-6 (such as Sepineo PHD 100), or combinations thereof. The cellulose-based polymer is selected from hydroxypropylcellulose (HPC, such as HPC-HF, HPC-JF, HPC-MF), hydroxyethylcellulose (HEC, such as Natrosol 250 HHX), hydroxypropyl methylcellulose (HPMC, such as Benecel E4M pharm), or combinations thereof. The poloxamer is selected from Ethylene oxide/propylene oxide copolymer (such as Poloxamer 188), triblock copolymer consisting of a central hydrophobic block of polypropylene glycol flanked by two hydrophilic blocks of polyethylene glycol (PEG) (such as Poloxamer 407), or combinations thereof. The high molecular weight PEG is selected from PEG 1500, PEG 3350, PEG 4000, or combinations thereof. The polyamide is selected from Polyamide-3 (such as OleoCraft polyamide HP-31 or OleoCraft polyamide MP-30), Polyamide-8 (such as OleoCraft polyamide LP-20), or combinations thereof. The siligel is selected from xanthan gum, lecitihin, sclerotium gum, pullulan, or combinations thereof.

In embodiments described herein, the topical pharmaceutical foam compositions contain liquid propellants or gas propellants. In certain embodiments, the liquid propellants are selected from the group consisting of hydro fluoro alkanes (HFA, such as HFA134a), hydro fluoro ethane (HFE), hydro fluoro olefine (HFO, such as HFO1234ze), propane, isobutane, butane, pentane, isopentane, dimethyl ether, and combinations thereof. In certain embodiments, the gas propellants are selected from the group consisting of nitrogen, nitrous oxide, carbon dioxide, and combinations thereof.

In embodiments described herein, the topical pharmaceutical foam compositions further comprise emollients. In certain embodiments, the emollient is selected from the group consisting of mineral oil, white soft paraffin, isopropyl palmitate, glycerin, propylene glycol, and combinations thereof.

In certain embodiments, a topical pharmaceutical foam composition comprises a base composition of about 1% tapinarof, about 71.87% water, about 2% glycerin, about 10% propylene glycol, about 4% Kolliwax CSA50, about 5.5% Kolliphor CS20, and about 5% medium chain triglycerides, and about 5% propane and about 5% butane of the total weight of the base composition as propellants.

In certain embodiments, a topical pharmaceutical foam composition comprises a base composition of about 1% tapinarof, about 67.44% water, about 2% glycerin, about 10% propylene glycol, about 3.76% Kolliwax CSA50, about 5.17% Kolliphor CS20, about 5% medium chain triglycerides, and about 5% isopropyl palmitate, and about 10% propane of the total weight of the base composition as a propellant.

In certain embodiments, a topical pharmaceutical foam composition comprises a base composition of about 1% tapinarof, about 69.37% water, about 2% glycerin, about 10% propylene glycol, about 4% Kolliwax CSA50, about 5.5% Kolliphor CS20, and about 5% medium chain triglycerides, and about 10% butane of the total weight of the base composition as a propellant.

In certain embodiments, a topical pharmaceutical foam composition comprises a base composition of about 1% tapinarof, about 69.87% water, about 2% glycerin, about 10% propylene glycol, about 4% Kolliwax CSA50, about 5.5% Kolliphor CS20, about 5% medium chain triglycerides, and about 2% non-ionic emulsifying wax (Polawax NF), and about 5% propane and about 5% butane of the total weight of the base composition as propellants.

PEG-Based Foams

In certain embodiments, a topical pharmaceutical foam composition comprises about 1% to about 4% tapinarof, about 6% to about 15% diethylene glycol monoethyl ether (DEGEE), about 10% to about 40% PEG 400, about 4% to about 10% propylene glycol, about 0.5% to about 2% benzyl alcohol, about 3% to about 15% PEG 4000, about 0.5% to about 2% of an emulsifier, and about 20% to about 50% of a propellant.

In certain embodiments, the emulsifier is selected from polyoxyethylene (2) stearyl ether (such as Brij S2), polyoxyethylene (20) stearyl ether (such as Brij S20), steareth 2, steareth 20, polysorbate 80, Polawax NF, glyceryl monostearate, or combination thereof.

In certain embodiments, the propellant is selected from hydro fluoro alkanes (HFA, such as HFA134a), hydro fluoro ethane (HFE), hydro fluoro olefine (HFO, such as HFO1234ze), propane, isobutane, butane, pentane, isopentane, dimethyl ether, and combinations thereof. In certain embodiments, the gas propellants are selected from the group consisting of nitrogen, nitrous oxide, carbon dioxide, and combinations thereof.

In certain embodiments, the topical pharmaceutical foam composition further comprises about 4% to about 10% water. In certain embodiments, the topical pharmaceutical foam composition further comprises about 8% to about 30% glycerol. In certain embodiments, the topical pharmaceutical foam composition further comprises about 5% to about 10% aluminum starch octenylsuccinate.

In certain embodiments, a topical pharmaceutical foam composition comprises about 1% tapinarof, about 7% water, about 11% diethylene glycol monoethyl ether (DEGEE), about 38.5% PEG400, about 7.4% propylene glycol, about 1.5% benzyl alcohol, about 12.6% PEG4000, about 1% Brij S2, and about 20% HFA134A as a propellant.

In certain embodiments, a topical pharmaceutical foam composition comprises about 1% tapinarof, about 4.38% water, about 6.88% diethylene glycol monoethyl ether (DEGEE), about 23.69% PEG400, about 4.63% propylene glycol, about 0.94% benzyl alcohol, about 7.88% PEG4000, about 0.63% Brij S2, and about 50% HFA134A as a propellant.

In certain embodiments, a topical pharmaceutical foam composition comprises about 1% tapinarof, about 10% glycerol, about 15% diethylene glycol monoethyl ether (DEGEE), about 29% PEG400, about 10% propylene glycol, about 1.5% benzyl alcohol, about 7.5% PEG4000, about 1% Brij S2, about 5% aluminum starch octenylsuccinate, and about 20% HFA134A as a propellant.

In certain embodiments, a topical pharmaceutical foam composition comprises about 1% tapinarof, about 10% glycerol, about 11% diethylene glycol monoethyl ether (DEGEE), about 29.88% PEG400, about 6.62% propylene glycol, about 1.5% benzyl alcohol, about 9% PEG4000, about 1% Brij S2, and about 30% HFA134A as a propellant.

In certain embodiments, a topical pharmaceutical foam composition comprises about 1% tapinarof, about 26.5% glycerol, about 12.5% diethylene glycol monoethyl ether (DEGEE), about 21.5% PEG400, about 8% propylene glycol, about 1.5% benzyl alcohol, about 8% PEG4000, about 1% Brij S2, about 10% aluminum starch octenylsuccinate, and about 20% HFA134A as a propellant.

In certain embodiments, a topical pharmaceutical foam composition comprises about 1% tapinarof, about 8.5% glycerol, about 15% diethylene glycol monoethyl ether (DEGEE), about 29% PEG400, about 10% propylene glycol, about 1.5% benzyl alcohol, about 4% PEG4000, about 1% Brij S2, and about 20% HFA134A as a propellant.

In certain embodiments, a topical pharmaceutical foam composition comprises about 1% tapinarof, about 7% water, about 11% diethylene glycol monoethyl ether (DEGEE), about 38.5% PEG400, about 7.4% propylene glycol, about 1.5% benzyl alcohol, about 12.6% PEG4000, about 1% Brij S20, and about 20% HFA134A as a propellant.

In certain embodiments, a topical pharmaceutical foam composition comprises about 1% tapinarof, about 8.5% glycerol, about 15% diethylene glycol monoethyl ether (DEGEE), about 29% PEG400, about 10% propylene glycol, about 1.5% benzyl alcohol, about 4% PEG4000, about 1% Brij S2, about 10% aluminum starch octenylsuccinate, and about 20% HFO1234ze as a propellant.

In certain embodiments, a topical pharmaceutical foam composition comprises about 1% tapinarof, about 8.5% glycerol, about 15% diethylene glycol monoethyl ether (DEGEE), about 29% PEG400, about 10% propylene glycol, about 1.5% benzyl alcohol, about 4% PEG4000, about 1% Brij S2, about 10% aluminum starch octenylsuccinate, and about 20% DME as a propellant.

Cream-Based Foams

In certain embodiments, a topical pharmaceutical foam composition comprises about 1% to about 4% tapinarof, about 30% to about 60% water, about 5% to about 20% propylene glycol, about 1% to about 15% of a solvent, about 1% to about 10% of an emulsifier, and about 20% of a propellant.

Embodiments described herein are directed to topical pharmaceutical foam compositions comprising a base composition of about 0.5% to about 2% tapinarof, about 85% to about 95% of a solvent, and about 3% to about 10% of an emulsifier, and about 5% to about 10% of the total weight of the base composition of a propellant.

In certain embodiments, the solvent is selected from water, ethanol, isopropanol, benzyl alcohol, phenoxyethanol, cetostearyl alcohol, cetyl alcohol, hexylene glycol, dipropylene glycol, butylene glycol, glycol, propylene glycol, diethylene glycol monoethyl ether (DEGEE, also known as Transcutol P), glycerol, dimethyl sulfoxide (DMSO), N-methyl-2-pyrrolidone (NMP), dimethyl isosorbide (DMI), medium chain triglycerides (such as Miglyol 812N), D-panthenol, isostearic acid (such as Prisorine 3505), liquid lanolin (such as liquid medilan ultra), oleic acid, PEG 10 dimethicone, or combinations thereof.

In certain embodiments, the emulsifier is selected from steareth 2, steareth 20, polysorbate 80, polyoxyethylene (2) stearyl ether (such as Brij S2), polyoxyethylene (20) stearyl ether (such as Brij S20), Polawax NF (non-ionic emulsifying wax), glyceryl monostearate, or combinations thereof.

In certain embodiments, the propellant is selected from hydro fluoro alkanes (HFA, such as HFA134a), hydro fluoro ethane (HFE), hydro fluoro olefine (HFO, such as HFO1234ze), propane, isobutane, butane, pentane, isopentane, dimethyl ether, and combinations thereof. In certain embodiments, the gas propellants are selected from the group consisting of nitrogen, nitrous oxide, carbon dioxide, and combinations thereof.

In certain embodiments, the topical pharmaceutical foam composition further comprises an emollient selected from mineral oil, white soft paraffin, isopropyl palmitate, or combinations thereof.

In certain embodiments, a topical pharmaceutical foam composition comprises about 1% tapinarof, about 31.5% water, about 15% propylene glycol, about 1% benzyl alcohol, about 3% Brij S20, about 4% glyceryl monostearate, about 1.5% cetostearl alcohol, about 11% mineral oil, about 4.5% white soft paraffin, about 7.5% isopropyl palmitate, and about 20% HFA134A as a propellant.

In certain embodiments, a topical pharmaceutical foam composition comprises about 1% tapinarof, about 51.85% water, about 0.15% sodium citrate dehydrate, about 0.07% citric acid monohydrate, about 0.08% EDTA, about 1.62% diethylene glycol monoethyl ether (DEGEE), about 8.08% propylene glycol, about 1.21% polysorbate 80, about 0.08% BHT, about 0.53% Brij S20, about 0.87% Brij S2, about 3.5% Polawax NF, about 10.75% Miglyol 810, and about 20% HFA134A as a propellant.

In certain embodiments, a topical pharmaceutical foam composition comprises a base composition of about 1% tapinarof, about 67.87% water, about 10% Miglyol 812N, about 1.8% steareth-2, about 1.1% steareth-20, about 1.5% polysorbate 80, about 4% Polawax NF, about 2% Transccutol P, and about 10% propylene glycol, and about 5% propane and 5% butane of the total weight of the base composition as propellants.

In certain embodiments, a topical pharmaceutical foam composition comprises a base composition of about 1% tapinarof, about 66.37% water, about 10% Miglyol 812N, about 1.8% steareth-2, about 1.1% steareth-20, about 1.5% polysorbate 80, about 4% Polawax NF, about 2% Transccutol P, and about 10% propylene glycol, and about 10% propane of the total weight of the base composition as a propellant.

In certain embodiments, a topical pharmaceutical foam composition comprises a base composition of about 1% tapinarof, about 67.87% water, about 10% Miglyol 812N, about 1.8% steareth-2, about 1.1% steareth-20, about 1.5% polysorbate 80, about 4% Polawax NF, about 2% Transccutol P, and about 10% propylene glycol, and about 10% HFA-134A of the total weight of the base composition as a propellant.

In certain embodiments, a topical pharmaceutical foam composition comprises a base composition of about 1% tapinarof, about 69.87% water, about 10% Miglyol 812N, about 1.8% steareth-2, about 1.1% steareth-20, about 1.5% polysorbate 80, about 2% Polawax NF, about 2% Transccutol P, and about 10% propylene glycol, and about 5% propane of the total weight of the base composition as a propellant.

In certain embodiments, a topical pharmaceutical foam composition comprises a base composition of about 1% tapinarof, about 66.87% water, about 10% Miglyol 812N, about 1.8% steareth-2, about 1.1% steareth-20, about 1.5% polysorbate 80, about 4% cetyl alcohol, about 2% Transccutol P, and about 10% propylene glycol, and about 10% propane of the total weight of the base composition as a propellant.

In certain embodiments, a topical pharmaceutical foam composition comprises a base composition of about 1% tapinarof, about 69.87% water, about 10% Miglyol 812N, about 1.8% steareth-2, about 1.1% steareth-20, about 1.5% polysorbate 80, about 2% Polawax NF, about 2% Transccutol P, and about 10% propylene glycol, and about 7.5% butane of the total weight of the base composition as a propellant.

Hybrid Emulsions

Embodiments described herein are directed to topical pharmaceutical emulsion compositions comprising: about 1% to about 4% tapinarof, about 15% glycerol, about 55% to about 60% water, about 10% isopropyl myristate, about 4% mixture of dimethicone and dimethiconol (such as dimethiconol blend 20), about 5% polydimethylsiloxane (such as TI-1050 fluid 350 Csp), and about 1.5% to about 10% of a gelling agent selected from Cyclopentasiloxane (and) Cyclohexasiloxane (and) Aluminum/Magnesium Hydroxide Stearate (such as Gilugel Sil 5), Sorbitan Sesquioleate (such as Span83), Hydroxyethyl Acrylate/Sodium Acryloyldimethyl Taurate Copolymer (such as Sepineo D.E.R.M.), or combination thereof.

Embodiments described herein are directed to topical pharmaceutical emulsion compositions comprising: about 1% to about 4% tapinarof, about 5% glycerol, about 20% diethylene glycol monoethyl ether (DEGEE) or propylene glycol, about 20% Mixture of Triceteareth-4 phosphate and Ethylene glycol palmitostearate and Diethylene glycol palmitostearate (such as Sedefos 75), about 5% Linoleoyl polyoxyl-6 glycerides NF/Linoleoyl macrogol-6 glycerides EP (such as Labrafil M 2125 CS), about 10% mineral oil, about 5% Glyceryl Behenate (such as Compritol 888), and about 35% Propylene glycol monolaurate (type II) EP/NF (such as Lauroglycol 90).

Dermatologically Acceptable Excipients

The topical pharmaceutical compositions described herein may further comprise one or more additional dermatologically acceptable excipients. Exemplary additional dermatologically acceptable excipients include, but are not limited to, an antioxidant, a pH adjusting agent, a chelating agent, a preservative, a co-solvent, a penetration enhancer, a humectant, a thickening or gelling or viscosity building agent, a fragrance, a colorant, and mixtures thereof.

Antioxidants

The topical pharmaceutical compositions described herein may further comprise an antioxidant. In an embodiment, the antioxidant is a mixture of two or more antioxidants.

Exemplary antioxidants include, but are not limited to, butylated hydroxytoluene (BHT), butylated hydroxyanisole (BHA), tocopherol, propyl gallate, vitamin E TPGS and tert-Butylhydroquinone (TBHQ), and mixtures thereof. In an embodiment, the antioxidant is selected from the group consisting of butylated hydroxytoluene, propyl gallate and tocopherol, and mixtures thereof.

In one embodiment, the antioxidant is butylated hydroxytoluene. In another embodiment, the antioxidant is propyl gallate. In yet another embodiment, the antioxidant is a mixture of butylated hydroxytoluene and propyl gallate.

In an embodiment, the antioxidant is used in conjunction with a chelating agent to prevent or minimize metal-catalyzed reactions, such as reactions catalyzed by iron, nickel, copper, magnesium, calcium, zinc or aluminum ions.

Suitably, the antioxidant is present in the composition in an amount from about 0.001% to about 5% by weight, based on the total weight of the composition. In an embodiment, the antioxidant is present in an amount from about 0.01% to 1% by weight, such as about 0.05% by weight or about 0.1% by weight, based on the total weight of the composition.

In one embodiment, the additional dermatologically acceptable excipient is a preservative. In one embodiment, the additional dermatologically acceptable excipient is at least one co-solvent. In one embodiment, the additional dermatologically acceptable excipient is selected from the group consisting of a pH adjusting agent, a chelating agent, a preservative and a co-solvent, and mixtures thereof. In another embodiment, the additional dermatologically acceptable excipient comprises a mixture of a pH adjusting agent, a chelating agent, a preservative and a co-solvent.

pH Adjusting Agent

The present topical pharmaceutical compositions may further comprise a pH adjusting agent.

In an embodiment, the pH adjusting agent is an acid, an acid salt, or a mixture thereof. Suitably, the acid is selected from the group consisting of lactic acid, acetic acid, maleic acid, succinic acid, citric acid, benzoic acid, boric acid, sorbic acid, tartaric acid, edetic acid, phosphoric acid, nitric acid, sulphuric acid and hydrochloric acid, and mixtures thereof.

In another embodiment, the pH adjusting agent is a buffer. Suitably, the buffer is selected from the group consisting of citrate/citric acid, acetate/acetic acid, phosphate/phosphoric acid, propionate/propionic acid, lactate/lactic acid, ammonium/ammonia and edetate/edetic acid. In one embodiment, the pH adjusting agent is a buffer which is citrate/citric acid.

Suitably, the pH adjusting agent is present in the composition in an amount from about 0.01% to about 10% by weight, based on the total weight of the composition. In an embodiment, the pH of the composition is adjusted with a pH adjusting agent to a pH of from about 4 to about 7, such as from about 4.5 to about 6.5.

Chelating Agents

The present topical pharmaceutical compositions may further comprise a chelating agent. In an embodiment, the chelating agent is a mixture of two or more chelating agents. As described herein, the compositions of the invention may comprise a mixture of a chelating agent and an antioxidant, where both excipients act to prevent or minimize oxidative degradation reactions in the composition.

Exemplary chelating agents include, but are not limited to, citric acid, glucuronic acid, sodium hexametaphosphate, zinc hexametaphosphate, ethylene diamine tetraacetic acid (EDTA), phosphonates, salts thereof, and mixtures thereof. Ethylene diamine tetraacetic acid is also known as edetic acid.

In one embodiment, the chelating agent is EDTA or a salt thereof, such as potassium, sodium or calcium salts of EDT A. In an embodiment, the EDTA or a salt thereof is disodium EDTA. In another embodiment, the chelating agent is citric acid. In yet another embodiment, the compositions of the invention comprise a mixture of a chelating agent and an antioxidant which is a mixture of EDTA or a salt thereof and propyl gal late. In a further embodiment, the compositions of the invention comprise a mixture of a chelating agent and an antioxidant which is a mixture of EDT A or a salt thereof and BHT. In one embodiment, the compositions of the invention comprise a mixture of a chelating agent and an antioxidant which is a mixture of disodium EDTA and BHT.

In yet a further embodiment, the compositions comprise a mixture of a chelating agent and an antioxidant which is a mixture of citric acid and propyl gallate. In an embodiment, the compositions of the invention comprise a mixture of a chelating agent and an antioxidant which is a mixture of citric acid and BHT.

Suitably, the chelating agent is present in the composition in an amount from about 0.01% to about 1% by weight, based on the total weight of the composition. In one embodiment, the chelating agent is present in the composition in an amount of about 0.1% by weight, based on the total weight of the composition.

Preservatives

The present topical pharmaceutical compositions may further comprise a preservative. In an embodiment, the preservative is a mixture of two or more preservatives.

Exemplary preservatives include, but are not limited to, benzyl alcohol, imidazolidinyl urea, diazolidinyl urea, dichlorobenzyl alcohol, chloroxylenol, methyl paraben, ethyl paraben, propyl paraben, butyl paraben, phenoxyethanol, sorbic acid, benzoic acid, salts thereof, and mixtures thereof.

In an embodiment, the preservative is selected from the group consisting of benzyl alcohol, phenoxyethanol and benzoic acid, and mixtures thereof.

In one embodiment, the preservative is benzyl alcohol. In another embodiment, the preservative is phenoxyethanol. In yet another embodiment, the preservative is benzoic acid.

Suitably, the preservative is present in the composition in an amount from about 0.01% to about 2% by weight, based on the total weight of the composition. In one embodiment, the preservative is present in the composition in an amount of about 0.25% by weight, based on the total weight of the composition.

Penetration Enhancer

The present topical pharmaceutical compositions may further comprise a penetration enhancer. In an embodiment, the penetration enhancer is a mixture of two or more penetration enhancers.

Exemplary penetration enhancers include, but are not limited to, fatty acids, fatty acid esters, fatty alcohols, pyrrolidones, sulfoxides, alcohols, diols and polyols, and mixtures thereof.

Exemplary fatty acids include, but are not limited to, oleic acid, capric acid, hexanoicacid, lauric acid, linoleic acid, linolenic acid, propionic acid and vaccenic acid, and mixtures thereof.

Exemplary fatty acid esters include, but are not limited to, glycerol monolaurate, glycerol monooleate, glycerol monolinoleate, isopropyl isostearate, isopropyl palmitate, isopropyl myristate, diethylsebacate, sorbitan monopalmitate, sorbitan oleate, sorbitan dilaurate, sorbitan trioleate, propylene glycol monolaurate and sucrose monolaurate, and mixtures thereof.

Exemplary fatty alcohols include, but are not limited to, cetyl alcohol, stearyl alcohol, decanol, tridecanol, lauryl alcohol, linolenyl alcohol and oleyl alcohol, and mixtures thereof.

Exemplary pyrrolidones include, but are not limited to, N-methyl pyrrolidone, 2-pyrrolidone and N-cyclohexyl-2-pyrrolidone, and mixtures thereof.

Exemplary sulfoxides include, but are not limited to, dimethyl sulfoxide and decylmethyl sulfoxide, and mixtures thereof.

Exemplary alcohols include, but are not limited to, lower (C1-C6) alcohols and diethylene glycol monoethyl ether, and mixtures thereof.

Exemplary diols include, but are not limited to, 1,2-hexanediol, butylene glycol, diethylene glycol, dipropylene glycol, ethyl hexanediol, ethylene glycol, hexylene glycol, pentylene glycol, propylene glycol, propylene glycol monolaurate, tetraethylene glycol, triethylene glycol, tripropylene glycol, polyethylene glycol and polypropylene glycol, and mixtures thereof.

Exemplary polyols include, but are not limited to, butanetriol, glycerol and 1,2,6-hexanetriol, and mixtures thereof.

Suitably, the penetration enhancer is present in the composition in an amount from about 0.5% to about 40% by weight, such as from about 1% to about 20% by weight or from about 5% to about 15% by weight, based on the total weight of the composition.

Combination with Other APIs

The present invention also provides for a pharmaceutical product comprising a combination of therapeutic agents, for simultaneous, separate or sequential use in the treatment of conditions for which administration of 3,5-Dihydroxy-4-isopropyl-trans-stilbene or a pharmaceutically acceptable salt thereof is indicated.

In the context of this specification, the term “simultaneously” when referring to simultaneous administration of the relevant drugs means at exactly the same time, as would be the case, for example in embodiments where the drugs are combined in a single preparation. In other embodiments, “simultaneously” can mean one drug is administered a short duration after another, wherein “a short duration” means a duration which allows the drugs to have their intended synergistic effect.

In light of the foregoing, the present invention also relates to combination therapy, which may be a comprised of a simultaneous or co-administration, or serial administration of a combination of compounds or pharmaceutical compositions of the present invention with other active drug or therapeutic agents, and where such administration also is determined by one of ordinary skill in the art.

In such an aforementioned combination composition, the dosage form of the present invention, each of the active drug components are contained in effective dosage amounts.

In another aspect, the present invention relates to a combination therapy, where the second therapeutic agent may be administered before, concurrent with or after administration of the 3,5-Dihydroxy-4-isopropyl-trans-stilbene or a pharmaceutically acceptable salt thereof whether in the same formulation or in a separate formulation and whether or not the second therapeutic agent is administered by the same topical route, e.g., it may be given orally, intravenously intramuscularly, ophthalmically, vaginally, rectally, etc.

In other words, the 3,5-Dihydroxy-4-isopropyl-trans-stilbene or a pharmaceutically acceptable salt thereof may be administered together, contemporaneously or sequentially in either order to the site of administration, or to a desired site of action. The order of administration is not deemed necessary, provided that if topically administered they are in contact at some point together at the site of administration or desired site of action. If both are present in the same vehicle they provide ease of administration to the patient, and perhaps increased compliance, but it is not required for the invention herein.

In another embodiment, the topical pharmaceutical compositions have greater than 90% of the original concentration of 3,5-Dihydroxy-4-isopropyl-trans-stilbene or a pharmaceutically acceptable salt thereof remaining after storage of the composition for 3 months at 40° C.

Applied Dose

When discussed in a biological function, the term applied dose may be used. As used herein, applied dose is defined as the amount of drug product applied per body surface area, denoted in mg/cm² units. The amount of active ingredient delivered to the skin layers (epidermis or dermis) may be denoted in nanograms (ng) or micrograms (μg) per skin section or per cm². Alternatively, the amount of active ingredient delivered to epidermis or dermis may be denoted as % of the applied dose. The amount of active ingredient delivered to the receiving fluid may be denoted as cumulative amount in ng or ng/cm².

In embodiments described herein, the topical composition has an in vitro skin permeation profile of about 10 ng/cm² to about 65 ng/cm².

In embodiments described herein, the topical composition has an in vitro penetration into the epidermis profile of about 500 ng to about 5,200 ng.

In embodiments described herein, the topical composition has an in vitro penetration into the dermis profile of about 1,800 ng to about 12,000 ng.

In an embodiment, the composition comprises 3,5-Dihydroxy-4-isopropyl-trans-stilbene or a pharmaceutically acceptable salt thereof in a composition that has a human skin penetration measured in vitro of at least 0.01-10% of the applied dose of the active ingredient into the epidermis over a period of about 1 to about 72 hours. In another embodiment, the time period is from about 2 to about 24 hours. In another embodiment, the time period is about 1 to about 15 hours. The % of applied dose of the active ingredient may be from 0.01-10%, 0.01-5%, 0.01-3%, 0.4-2.3% w/w.

In one embodiment, the composition comprises the 3,5-Dihydroxy-4-isopropyl-trans-stilbene or a pharmaceutically acceptable salt thereof in a composition that has human skin penetration measured in vitro of at least 0.01-10% of the applied dose of the active ingredient into the dermis over a period of about 1 to about 72 hours. In another embodiment, the time period is from about 2 to about 24 hours. In another embodiment, the time period is about 6 to about 15 hours. The % of applied dose of the active ingredient may be from 0.01-7.5%, 0.01-5%, 0.01-3%, 0.3-1.7%. In an alternative embodiment, the applied dose measured in an amount of 1-2 μg/cm², e.g., 0.5% w/v.

One embodiment of the invention is a topical pharmaceutical composition described herein wherein the composition administered in an in vitro system results in a ratio of dermis amounts (ng) measured at steady state to normalized (by active strength) skin flux (ng*cm2/hr) from 1000 to 5000, using freshly excised abdominal human skin. In one embodiment, the 3,5-Dihydroxy-4-isopropyl-trans-stilbene or pharmaceutically acceptable salt thereof is solubilized in the oil phase of the emulsion composition.

In another embodiment of the invention, the topical pharmaceutical composition described herein minimizes the area under the curve (AUC) AUC(0-tau) in a human upon administration to the skin in an amount not exceeding 35% Body Surface Area (BSA). In another embodiment, the amount is not exceeding 30% BSA.

In one embodiment, the AUC is at steady state. In another embodiment, the amount of body surface area (BSA) for which the drug is applied to is less than 50%, in another embodiment the amount is less than 35%, in another embodiment the amount is less than 30%. It is recognized that if the BSA is >10% than the AUC may be increased accordingly.

As used herein, the term “AUC(0-tau)” means the area under the plasma concentration versus time curve from time 0 to end of the dosing interval, as calculated by the log-linear trapezoidal method.

Methods of Use

Embodiments are directed to methods of treating a dermatological condition or disorder in a patient in need thereof, the method comprising topically administering to an affected area of said patient a topical pharmaceutical composition as described herein.

Another aspect of the invention is the use of a topical pharmaceutical composition described herein in the manufacture of a medicament for the treatment of dermatological condition or disorder in a patient.

In one embodiment, the dermatological disease or disorder is atopic dermatitis, psoriasis or acne.

The compositions of the present invention may be used in a veterinary setting or in a medical setting, topically. It is recognized that the patient or subject may be an animal, a domestic animal, such as a mammal, including horses, cows, pigs, sheep, poultry, fish, cats, dogs and zoo animals. In one embodiment, the patient is an animal. In another embodiment, the patient is a mammal. In another embodiment, the mammal is a human. In another embodiment, the human is an adult, or a pediatric patient. In one embodiment, the pediatric patient is a child. In another embodiment, the pediatric patient is 3 months to 2 years of age and older.

In one embodiment, the dermatological condition or disorder for which treatment is sought is an inflammatory skin disease (e.g., a chronic inflammatory skin disease such as dermatitis (e.g., atopic dermatitis, contact dermatitis, eczematous dermatitis, or seborrhoic dermatitis), acne, psoriasis, rosacea, or aging skin.

In some aspects, the dermatological condition or disorder is selected from the group for the treatment of a skin disease, wherein the skin disease comprises a skin disorder of persistent inflammation, cell kinetics, and differentiation (e.g., psoriasis, psoriatic arthritis, exfoliative dermatitis, Pityriasis rosea, Lichen planus, Lichen nitidus, or porokeratosis); a skin disorder of epidermal cohesion, vesicular and bullous disorders (e.g., pemphigus, bulluous pemphigoi, epidermamolysis bullosa acquisita, or pustular eruptions of the palms or soles); a skin disorder of epidermal appendages and related disorders (e.g., hair disorders, nails, rosacea, perioral dermatitis, or follicular syndromes); a skin disorder such as an epidermal and appendageal tumors (e.g., squamous cell carcinoma, basal cell carcinoma, keratoacanthoma, benign epithelial tumors, or merkel cell carcinoma); a disorder of melanocytes (e.g., pigmentary disorders, albinism, hypomelanoses and hypermelanoses, melanocytic nevi, or melanoma); a skin disorder of inflammatory and neoplastic disorders of the dermis (e.g., erythema elavatum diutinum, eosinophils, granuloma facilae, pyoderma gangrenosum, malignant atrophic papulosis, fibrous lesions of dermis and soft tissue, or Kaposi sarcoma); a disorder of the subcutaneous tissue (e.g., panninculitis or lipodystrophy); a skin disorder involving cutaneous changes of altered reactivity (e.g., urticaria, angiodererma, graft-vs-host, allergic contact dermatitis, autosensitization dermatitis, atopic dermatitis, radiation dermatitis, or seborrheic dermatitis); a skin change due to mechanical and physical factors (e.g., thermal injury, radiation dermatitis, corns, or calluses); photodamage (e.g., acute and chronic UV radiation, or photosensitization); or a skin disorder due to microbial agents (e.g., leprosy, lyme borreliosis, onychomycosis, tinea pedra, rubella, measles, herpes simplex, EBY (Epstein-Barr virus), HPV (Human papillomavirus, e.g., HPV6 & 7), warts, or prions).

In one embodiment, the inflammatory disorder is selected from the group consisting of psoriasis, and atopic dermatitis and acne. In an embodiment, the dermatological condition or disorder is psoriasis. In another embodiment, the dermatological condition or disorder is atopic dermatitis. In another embodiment, the dermatological condition or disorder is acne. In another embodiment, the dermatological condition or disorder is radiation dermatitis. In another embodiment, the dermatological condition or disorder is inverse psoriasis. In another embodiment, the dermatological condition or disorder is hand eczema. In another embodiment, the dermatological condition or disorder is hidradenitis suppurativa or acne inversa.

Embodiment 1 is directed to an aqueous gel compositions comprising:

about 1% to about 4% tapinarof,

about 10% to about 65% water,

about 10% to about 50% diethylene glycol monoethyl ether (DEGEE),

about 5% to about 65% of a glycol,

about 2% to about 55% of a solvent,

about 0.5% to about 5% of a gelling agent, and

a neutralizing agent.

Embodiment 2 is directed to an anhydrous gel compositions comprising:

about 1% to about 4% tapinarof,

about 10% to about 70% of a solvent,

about 10% to about 30% diethylene glycol monoethyl ether (DEGEE),

about 15% to about 50% of a glycol, and

about 0.5% to about 5% of a gelling agent.

Embodiment 3 is directed to the composition of embodiment 1, wherein the composition has a pH of about 6 to about 6.5.

Embodiment 4 is directed to the composition of embodiments 1 or 2, wherein the solvent is selected from the group consisting of N-methyl-2-pyrrolidone (NMP), dimethyl sulfoxide (DMSO), dimethyl isosorbide (DMI), benzyl alcohol, phenoxyethanol, ethanol, cetostearyl alcohol, glycerol, medium chain triglycerides, D-panthenol, isostearic acid (such as Prisorine 3505), liquid medilan ultra, oleic acid, PEG 10 dimethicone, and combinations thereof.

Embodiment 5 is directed to the composition of embodiments 1 or 2, wherein the glycol is selected from the group consisting of low molecular weight PEG, PEG 200, PEG 300, PEG 400, propylene glycol, dipropylene glycol, hexylene glycol, butylene glycol, and combinations thereof.

Embodiment 6 is directed to the composition of embodiments 1 or 2, wherein the gelling agent is selected from the group consisting of a carbomer, an acrylate-based polymer, a cellulose-based polymer, a poloxamer, a high molecular weight PEG, a polyamide, a siligel, and combinations thereof.

Embodiment 7 is directed to the composition of embodiment 1, wherein the neutralizing agent is selected from the group consisting of sodium hydroxide, ammonium hydroxide, potassium hydroxide, arginine, aminomethyl propanol, tetrahydroxypropyl ethylenediamine, triethanolamine, tromethamine, PEG-15 cocamine, diisopropanolamine, triisopropanolamine, trolamine, and combinations thereof.

Embodiment 8 is directed to an ointment composition comprising:

about 1% to about 4% tapinarof,

about 10% to about 70% of a low molecular weight PEG,

about 50% to about 75% of a solvent, and

about 25% to about 35% of a high molecular weight PEG.

Embodiment 9 is directed to an ointment composition embodiment 8, wherein the low molecular weight PEG is selected from the group consisting of PEG 200, PEG 300, PEG 400, PEG 500, PEG 600, and combinations thereof.

Embodiment 10 is directed to an ointment composition embodiment 8, wherein the solvent is selected from the group consisting of a glycol, diethylene glycol monoethyl ether (DEGEE), an alcohol, water, glycerol, dimethyl sulfoxide (DMSO), N-methyl-2-pyrrolidone (NMP), dimethyl isosorbide (DMI), medium chain triglycerides, D-panthenol, isostearic acid (such as Prisorine 3505), liquid lanolin (such as liquid medilan ultra), oleic acid, PEG 10 dimethicone, and combinations thereof.

Embodiment 11 is directed to an ointment composition embodiment 10, wherein the glycol is selected from the group consisting of propylene glycol, hexylene glycol, dipropylene glycol, butylene glycol, and combinations thereof.

Embodiment 12 is directed to an ointment composition embodiment 10, wherein the alcohol is selected from the group consisting of ethanol, isopropanol, benzyl alcohol, phenoxyethanol, cetostearyl alcohol, and combinations thereof.

Embodiment 13 is directed to an ointment composition embodiment 10, wherein the glycols are present in an amount of about 10% to about 40%.

Embodiment 14 is directed to an ointment composition embodiment 10, wherein the diethylene glycol monoethyl ether (DEGEE) is present in an amount of about 10% to about 40%.

Embodiment 15 is directed to an ointment composition embodiment 10, wherein the alcohol is present in an amount of about 1% to about 10%.

Embodiment 16 is directed to an ointment composition embodiment 10, wherein the water is present in an amount of about 1% to about 25%.

Embodiment 17 is directed to an ointment composition embodiment 10, wherein the glycerol is present in an amount of about 1% to about 25%.

Embodiment 18 is directed to an ointment composition embodiment 10, wherein the dimethyl sulfoxide (DMSO) is present in an amount of about 1% to about 50%.

Embodiment 19 is directed to an ointment composition embodiment 8, wherein the high molecular weight PEG is selected from the group consisting of PEG 1500, PEG 3350, PEG 4000, and combinations thereof.

Embodiment 20 is directed to an ointment composition embodiment 8, wherein the ointment composition further comprises about 10% silicone-based solvent.

Embodiment 21 is directed to a foam composition comprising:

about 1% to about 4% tapinarof,

about 20% to about 80% of a solvent,

about 0.5% to about 10% of an emulsifier,

about 5% to about 15% of a thickener, and

about 20% to about 50% of a propellant.

Embodiment 22 is directed to a foam composition of embodiment 21, wherein the solvent is selected from the group consisting of a glycol, diethylene glycol monoethyl ether (DEGEE), an alcohol, water, glycerol, dimethyl sulfoxide (DMSO), N-methyl-2-pyrrolidone (NMP), dimethyl isosorbide (DMI), medium chain triglycerides, D-panthenol, isostearic acid (such as Prisorine 3505), liquid lanolin (such as liquid medilan ultra), oleic acid, PEG 10 dimethicone, and combinations thereof.

Embodiment 23 is directed to a foam composition of embodiment 22, wherein the glycol is selected from the group consisting of low molecular weight PEG, PEG 200, PEG 300, PEG 400, propylene glycol, dipropylene glycol, hexylene glycol, butylene glycol, and combinations thereof.

Embodiment 24 is directed to a foam composition of embodiment 22, wherein the alcohol is selected from the group consisting of ethanol, isopropanol, benzyl alcohol, phenoxyethanol, cetostearyl alcohol, and combinations thereof.

Embodiment 25 is directed to a foam composition of embodiment 21, wherein the emulsifiers are selected from the group consisting of steareth 2, steareth 20, polysorbate 80, polyoxyethylene (2) stearyl ether (such as Brij S2), polyoxyethylene (20) stearyl ether (such as Brij S20), Polawax NF, glyceryl monostearate, and combinations thereof.

Embodiment 26 is directed to a foam composition of embodiment 21, wherein the thickeners are selected from the group consisting of a carbomer, an acrylate-based polymer, a cellulose-based polymer, a poloxamer, a high molecular weight PEG, a polyamide, a siligel, aluminum starch octenylsuccinate, and combinations thereof.

Embodiment 27 is directed to a foam composition of embodiment 21, wherein the propellants are selected from the group consisting of hydro fluoro alkanes (HFA), hydro fluoro ethane (HFE), hydro fluoro olefine (HFO), propane, isobutane, butane, pentane, isopentane, dimethyl ether, and combinations thereof.

Embodiment 28 is directed to a foam composition of embodiment 21, further comprising an emollient selected from the group consisting of mineral oil, white soft paraffin, isopropyl palmitate, glycerin, propylene glycol, and combinations thereof.

Embodiment 29 is directed to a foam composition comprising a base composition of:

about 1% to about 2% tapinarof,

about 80% to about 90% of a solvent, and

about 8% to about 15% of an emulsifier, and

about 5% to about 10% of the total weight of the base composition a propellant.

Embodiment 30 is directed to a foam composition comprising a base composition of:

about 0.5% to about 2% tapinarof,

about 85% to about 95% of a solvent, and

about 3% to about 10% of an emulsifier, and

about 5% to about 10% of the total weight of the base composition a propellant.

A method of treating a dermatological condition or disorder in a patient in need thereof, the method comprising administering to said patient the topical pharmaceutical composition of any of the previous embodiments.

The examples set forth below are illustrative of the present invention and are not intended to limit, in any way, the scope of the present invention. The following examples illustrate the invention. These examples are not intended to limit the scope of the present invention, but rather to provide guidance to the skilled artisan to prepare and use the compounds, compositions, and methods of the present invention.

### EXAMPLES

### Example 1: Excipient Studies

Solubility of tapinarof in a variety of excipients has been tested and is presented in Tables 1 and 2.

TABLE 1
Tapinarof saturated solubility results

| Excipient | Solubility (mg/g) |
|---|---|
| Water | 0.14 |
| Mineral oil | 0.26 |
| Glycerol | 14 |
| Sorbitan oleate | 137 |
| Diisopropyl adipate | 187 |
| Benzyl alcohol | 193 |
| Isopropyl palmitate | 231 |
| Polysorbate 80 | 263 |
| Medium chain triglycerides | 273 |
| Polyethylene glycol 400 | 373 |
| Phenoxyethanol | 381 |
| Hexylene glycol | 386 |
| Propylene glycol | 444 |
| Isopropyl alcohol | 485 |
| Diethyl sebacate | 488 |
| Dimethyl isosorbide | 496 |
| Propylene carbonate | 506 |
| diethylene glycol monoethyl ether | 526 |
| (DEGEE) | |

TABLE 2
Saturated solubility of tapinarof in individual excipients,
determined by tapinarof assay HPLC or visually

| | Average saturated solubility of DMVT- |
|---|---|
| Excipient | 505 (% w/w, mean of n = 3 (range)) |
| Castor oil | 7.06 |
| | (6.07-8.14) |
| Isopropyl myristate | ≥20 (visual solubility) |
| Kollicream ® OD | 11.03 |
| (octyldodecanol) | (11.01-11.06) |
| Kollicream ® OA | 15.82 |
| (oleyl alcohol) | (15.40-16.06) |
| Kollicream ® 3C | 19.03 |
| (cocoyl caprylocaprate) | (18.66-19.33) |
| Sunflower oil | 12.05 |
| | (11.84-12.17) |
| Ethanol | ≥20 (visual solubility) |
| Dipropylene glycol | ≥20 (visual solubility) |
| MethoxyPEG 350 | ≥20 (visual solubility) |
| Jojoba oil | 8.19 |
| | (8.01-8.43) |
| Safflower oil | 11.68 |
| | (11.51-11.83) |
| Sesame oil | 11.56 |
| | (11.54-11.57) |
| Olive oil | 11.35 |
| | (11.31-11.38) |
| Almond oil | 11.94 |
| | (11.79-12.02) |
| PEG 200 | ≥20% (visual solubility) |
| Aloe vera juice | 0.01 |
| | (0.00-0.01) |
| Walnut oil | 12.43 |
| | (12.14-12.93) |
| Soyabean oil | 11.68 |
| | (11.64-11.71) |

The stability of tapinarof in each excipient was also tested and is presented in Tables 3 and 4.

TABLE 3
Excipient stability of tapinarof in individual excipients, determined by
tapinarof assay HPLC method

| | Percentage of recovery of DMVT-505 at t = 0 and following 2 weeks of storage | | |
|---|---|---|---|
| | | t = 2 weeks | |
| Excipient | t = 0 | 40° C. | 70° C. |
| Castor oil | 98.86 (98.54-99.17) | 102.05 (101.22-102.89) | 98.30 (97.92-98.67) |
| IPM | 101.41 (101.26-101.55) | 98.38 (98.24-98.52) | 102.19 (101.64-102.75) |
| Kollicream ® | 100.20 (100.01-100.40) | 94.16 (93.41-94.92) | 83.50 (83.36-83.64) |
| OD | | | |
| Kollicream ® | 96.55 (96.42-96.67) | 95.27 (95.27-95.27) | 88.33 (88.11-88.55) |
| OA | | | |
| Diisopropyl | 99.55 (99.31-99.80) | 94.05 (90.03-98.07) | 92.84 (92.74-92.95) |
| adipate | | | |
| Kollicream ® | 99.73 (98.91-100.55) | 98.50 (98.35-98.65) | 96.68 (96.58-96.78) |
| 3C | | | |
| Sunflower oil | 98.29 (94.81-101.77) | 100.74 (99.56-101.92) | 99.78 (99.78-99.79) |
| Sesame oil | 101.19 (101.18-101.21) | 99.66 (98.97-100.34) | 97.78 (97.06-98.51) |
| Olive oil | 98.54 (98.36-98.71) | 98.39 (98.02-98.76) | 98.03 (98.00-98.07) |
| Almond oil | 93.05 (93.02-93.09) | 98.31 (97.60-99.02) | 95.43 (94.90-95.96) |
| IPP | 98.02 (97.37-98.67) | 96.86 (96.72-97.00) | 95.26 (95.16-95.36) |
| Jojoba oil | 102.54 (100.22-104.86) | 99.85 (99.49-100.20) | 97.42 (96.91-97.92) |
| Crodamol | 96.27 (92.36-100.18) | 88.46 (87.70-89.21) | 86.96 (86.83-87.09) |
| GTCC | | | |
| Dipropylene | 96.98 (96.87-97.09) | 96.11 (94.89-97.34) | 94.01 (93.73-94.30) |
| glycol | | | |
| MethoxyPEG | 99.10 (99.10-99.11) | 93.19 (90.07-96.32) | 83.26 (83.03-83.49) |
| 350 | | | |
| PEG 200 | 98.31 (98.23-98.39) | 100.01 (99.18-100.84) | 93.35 (93.25-93.45) |
| Ethanol | 99.60 (99.60-99.60) | 95.90 (95.54-96.26) | 95.26 (95.13-95.38) |
| PEG 400 | 96.70 (96.59-96.81) | 98.94 (98.38-99.50) | 90.93 (90.80-91.06) |
| Hexylene | 99.76 (99.40-100.11) | 98.28 (98.27-98.30) | 95.89 (95.61-96.18) |
| glycol | | | |
| IPA | 97.33 (96.92-97.75) | 96.10 (95.81-96.40) | 97.33 (95.03-99.64) |
| Propylene | 102.36 (102.01-102.72) | 100.03 (99.90-100.17) | 105.81 (105.52-106.09) |
| glycol | | | |
| Arlasolve DMI | 97.85 (97.84-97.87) | 87.71 (87.41-88.01) | 76.41 (76.14-76.68) |
| Diethyl | 101.25 (101.15-101.35) | 98.15 (97.91-98.39) | 95.58 (95.32-95.84) |
| sebacate | | | |
| Sorbitan | 86.17 (85.63-86.71) | 103.33 (103.32-103.34) | 95.36 (95.06-95.67) |
| monooleate | | | |
| (Span 80) | | | |

TABLE 4
Purity of tapinarof (as % area) in
individual excipients, determined by
tapinarof related substances HPLC method

| | Purity of DMVT-505 at t = 0 and | | |
|---|---|---|---|
| | following 2 weeks of storage (% area) | | |
| | | | t = 2 weeks |
| Excipient | t = 0 | 40° C. | 70° C. |
| Castor oil | 100.00 | 100.00 | 99.92 |
| IPM | 100.00 | 99.32 | 92.15 |
| Kollicream ® | 100.00 | 99.38 | 98.11 |
| OD | | | |
| Kollicream ® | 100.00 | 99.66 | 99.46 |
| OA | | | |
| Diisopropyl | 100.00 | 99.79 | 95.94 |
| adipate | | | |
| Kollicream ® | 100.00 | 98.98 | 96.51 |
| 3C | | | |
| Sunflower oil | 100.00 | 100.00 | 99.51 |
| Sesame oil | 100.00 | 99.94 | 99.67 |
| Olive oil | 100.00 | 99.91 | 99.74 |
| Almond oil | 100.00 | 99.96 | 99.84 |
| IPP | 100.00 | 99.30 | 99.52 |
| Jojoba oil | 99.97 | 98.98 | 99.09 |
| Crodamol | 99.92 | 96.46 | 96.94 |
| GTCC | | | |
| Dipropylene | 99.97 | 99.83 | 98.89 |
| glycol | | | |
| MethoxyPEG | 99.59 | 98.42 | 95.50 |
| 350 | | | |
| PEG 200 | 99.91 | 99.86 | 98.56 |
| Ethanol | 99.89 | 99.72 | 98.57 |
| PEG 400 | 99.87 | 99.30 | 98.63 |
| Hexylene | 99.96 | 99.54 | 98.76 |
| glycol | | | |
| IPA | 99.91 | 99.86 | 99.25 |
| Propylene | 99.90 | 99.51 | 99.23 |
| glycol | | | |
| Arlasolve DMI | 99.55 | 96.75 | 95.14 |
| Diethyl | 100.00 | 96.72 | 95.78 |
| sebacate | | | |
| Sorbitan | 100.00 | 100.00 | 99.79 |
| monooleate | | | |
| (Span 80) | | | |

### Example 2: Aqueous Gel (Hydrogel) Formulation Development

Tables 5A and 5B provide the aqueous gel formulations tested for drug concentration using HPLC, stability utilizing the following tests: macroscopic appearance, microscopic appearance, physical stability and chemical stability at 25° C./65% RH and 40° C./75% RH, and viscosity using a Brookfield viscometer.

TABLE 5A
Set 1 of Aqueous Gel Formulations

| Excipients | AG02 ACT* | AG05 ACT | AG12 ACT | AG15 ACT* | AG18 ACT* | AG25 ACT | AG33 ACT* |
|---|---|---|---|---|---|---|---|
| Solvent system | SSAG01 | SSAG01 | SSAG01 | SSAG06 | SSAG07 | SSAG12 | SSAG04 |
| DMVT-505/tapinarof | 2.40 | 2.40 | 2.40 | 1.00 | 1.80 | 4.00 | 1.00 |
| Water diethylene | 27.60 | 27.60 | 41.10 | 25.00 | 25.00 | 5.00 | 30.00 |
| glycol monoethyl ether (DEGEE) | 21.00 | 21.00 | 20.00 | 20.00 | 20.00 | 20.00 | 20.00 |
| PEG 400 | 10.00 | 10.00 | 10.00 | 10.00 | 10.00 | 40.00 | 10.00 |
| Propylene glycol | 23.00 | 23.00 | 23.00 | | | 23.00 | |
| Dipropylene glycol | | | | | | | |
| N-Methy1-2-pyrrolidone (NMP) | | | | 23.00 | | | |
| Dimethyl sulfoxide (DMSO) | | | | | 23.00 | | |
| Dimethyl isosorbide (DMI) | | | | | | | 23.00 |
| Benzyl alcohol | | | 2.00 | | | 2.00 | |
| Phenoxyethanol | 1.00 | 1.00 | | 1.00 | 1.00 | | 1.00 |
| crosslinked polyacrylic acid | 1.00 | | | 1.00 | 1.00 | 1.00 | 1.00 |
| (such as Carbopol ® 980) | | | | | | | |
| crosslinked copolymer of acrylic | | 1.00 | | | | | |
| acid and a hydrophobic C10-30 | | | | | | | |
| alkyl acrylate co-monomer (such | | | | | | | |
| as Pemulen TR-1 or Pemulen TR-2) | | | | | | | |
| Hydroxyethyl Acrylate/Sodium | | | 1.50 | | | | |
| Acryloyldimethyl Taurate Copolymer | | | | | | | |
| (such as Sepineo D.E.R.M.) | | | | | | | |
| HPC HF | | | | | | | |
| Natrosol 250 HHX (HEC) | | | | | | | |
| Benecel E4M pharm | | | | | | | |
| 0.2M NaOH | q.s pH 6-6.5 | | | q.s pH 6-6.5 | | | |
| Water 2nd addition | q.s 100% | | | q.s 100% | | | |
| Total | 86.00 | 86.00 | 100.00 | 81.00 | 81.80 | 95.00 | 86.00 |

TABLE 5B
Set 2 of Aqueous Gel Formulations

| | AG40 | AG41 | AG43 | AG47 | AG49 | AG50 |
|---|---|---|---|---|---|---|
| Excipients | ACT* | ACT* | ACT* | ACT | ACT | ACT |
| Solvent system | SSAG16 | N/A | N/A | SSAG02 | SSAG01 | SSAG15 |
| DMVT-505/tapinarof | 1.00 | 1.00 | 2.00 | 4.00 | 2.40 | 0.10 |
| Water | 30.00 | 20.00 | 40.00 | 30.00 | 41.60 | 58.90 |
| diethylene glycol monoethyl | 20.00 | 12.80 | 49.10 | 20.00 | 20.00 | 15.00 |
| ether (DEGEE) | | | | | | |
| PEG 400 | 23.00 | 6.40 | | 10.00 | 10.00 | 10.00 |
| Propylene glycol | | | | 23.00 | 13.00 | |
| Dipropylene glycol | | | | 23.00 | | |
| N-Methyl-2-pyrrolidone | | | | | | |
| (NMP) | | | | | | |
| Dimethyl sulfoxide (DMSO) | 10.00 | 50.00 | | | | |
| Dimethyl isosorbide (DMI) | | | | | | |
| Benzyl alcohol | | | | 2.00 | 2.00 | 2.00 |
| Phenoxyethanol | 1.00 | 1.00 | 1.00 | | | |
| crosslinked polyacrylic acid | 1.00 | 1.00 | 1.00 | 1.00 | | |
| (such as Carbopol +200 980) | | | | | | |
| crosslinked copolymer of | | | | | | |
| acrylic acid and a hydrophobic | | | | | | |
| C-10-30 alkyl arylate co- | | | | | | |
| monomer (such as Pemulen | | | | | | |
| TR-1 or Pemulen TR-2) | | | | | | |
| Hydroxyethyl Acrylate/Sodium | | | | | | |
| Acryloyldimethyl Taurate | | | | | | |
| Copolymer (such as Sepineo | | | | | | |
| D.E.R.M.) | | | | | | |
| HPC HF | | | | | | |
| Natrosol 250 HHX (HEC) | | | | | 1.00 | 1.00 |
| Benecel E4M pharm | | | | | | |
| 0.2M NaOH | | | | | | q.s pH 6-6.5 |
| Water 2nd addition | | | | | | q.s 100% |
| Total | 86.00 | 92.20 | 93.10 | 90.00 | 100.00 | 100.00 |

Following formulation stability, all of the developed aqueous gel formulations exhibited comparable physical and chemical stability. The following parameters (represented with * above) are recommended for evaluation in IVPT to assess impact on in vitro skin permeation: AG02 (23% PG), AG15 (23% NMP), AG18 (23% DMSO), AG33 (23% DMI), AG40 (10% DMSO), AG41 (50% DMSO) and AG43 (49% diethylene glycol monoethyl ether (DEGEE).

The API (tapinarof) was included in a range between 1-2.4% w/w in the gels. It should be noted that drug loading in formulation with different penetration enhancers is slightly different owing to the different solubility of the drug in the solvent system. However, these can still be compared on the basis of the % of the applied dose.

### Example 3: Anhydrous (Non-Aqueous) Gel Formulation Development

Tables 6A and 6B provide the anhydrous gel formulations tested for drug concentration using HPLC, stability utilizing the following tests: macroscopic appearance, microscopic appearance, physical stability and chemical stability at 25° C./65% RH and 40° C./75% RH, and viscosity using a Brookfield viscometer.

TABLE 6A
Set 1 of Anhydrous Gel Formulations

| | NA01 ACT | NA01 ACT | NA03 | NA06 | NA07 |
|---|---|---|---|---|---|
| Component | 1.0%* | 4.0%* | ACT | ACT* | ACT* |
| Solvents system | SSNA02 | SSNA02 | SSNA02 | SSNA06 | SSNA07 |
| DMVT-505 | 1.00 | 4.00 | 1.00 | 1.00 | 1.00 |
| diethylene glycol | 20.00 | 20.00 | 20.00 | 25.00 | 25.00 |
| monoethyl ether | | | | | |
| (DEGEE) | | | | | |
| Glycerol | 20.00 | 20.00 | 20.00 | 20.00 | 20.00 |
| PEG 400 | — | — | — | 42.00 | 42.00 |
| PEG 200 | 33.00 | 28.00 | 32.00 | — | — |
| Hexylene glycol | — | — | — | — | — |
| Propylene glycol | 15.00 | 15.00 | 15.00 | — | — |
| Ethanol | 10.00 | 10.00 | 10.00 | — | — |
| N-Methyl-2- | — | — | — | 10.00 | — |
| pyrrolidone | | | | | |
| (NMP) | | | | | |
| Dimethyl | — | — | — | — | 10.00 |
| sulfoxide (DMSO) | | | | | |
| HPC-HF | 1.00 | 1.00 | — | — | — |
| HPC-JF | — | — | — | — | — |
| HPC-MF | — | — | 2.00 | 2.00 | 2.00 |
| carboxypolymethylene | — | — | — | — | — |
| and carbomers (such | | | | | |
| as Carbopol ® 974) | | | | | |
| Trolamine | — | — | — | — | — |
| PEG 200 | — | — | — | — | — |
| Total | 100.00 | 100.00 | 100.00 | 100.00 | 100.00 |

TABLE 6B
Set 2 of Anhydrous Gel Formulations

| | NA09 | NA11 | NA12 | NA13 | OG04 |
|---|---|---|---|---|---|
| Component | ACT* | ACT* | ACT | ACT | ACT* |
| Solvents system | SSNA08 | SSNA07 | SSNA03 | SSNA01 | SSOG04 |
| DMVT-505 | 1.00 | 1.00 | 1.00 | 1.00 | 1.00 |
| diethylene | 20.84 | 13.63 | 20.00 | 20.00 | — |
| glycol | | | | | |
| monoethyl ether | | | | | |
| (DEGEE) | | | | | |
| Glycerol | 40.68 | 10.91 | 20.00 | 20.00 | — |
| PEG 400 | — | 23.45 | — | 32.00 | — |
| PEG 200 | 10.42 | — | 32.00 | — | — |
| Hexylene glycol | — | — | 15.00 | — | — |
| Propylene glycol | 15.63 | — | — | 15.00 | — |
| Ethanol | 10.42 | — | 10.00 | 10.00 | — |
| N-Methyl-2- | — | — | — | — | — |
| pyrrolidone | | | | | |
| (NMP) | | | | | |
| Dimethyl | — | 50.00 | — | — | — |
| sulfoxide | | | | | |
| (DMSO) | | | | | |
| HPC-HF | — | — | — | — | — |
| HPC-JF | — | — | — | — | — |
| HPC-MF | — | 2.00 | 2.00 | 2.00 | — |
| carboxypoly- | 1.00 | — | — | — | — |
| methylene and | | | | | |
| carbomers (such | | | | | |
| as Carbopol ® | | | | | |
| 974) | | | | | |
| Trolamine | q.s pH | — | — | — | — |
| | 6.0-6.5 | | | | |
| PEG 200 | q.s 100% | — | — | — | — |
| PPG-15 stearyl | — | — | — | — | 15.00 |
| ether | | | | | |
| Mineral oil (Light | — | — | — | — | 24.00 |
| mineral) | | | | | |
| Castor oil | — | — | — | — | 15.00 |
| Isopropyl | — | — | — | — | 15.00 |
| myristate | | | | | |
| Kollicream ® OD | — | — | — | — | 15.00 |
| (octyldodecanol) | | | | | |
| Myristyl lactate | — | — | — | — | 10.00 |
| Polyamide-8 | — | — | — | — | 10.00 |
| (such as | | | | | |
| OleoCraft | | | | | |
| polyamide LP-20) | | | | | |
| Total | 100.00 | 100.00 | 100.00 | 100.00 | 100.00 |

Following formulation stability, all of the developed non-aqueous gel formulations exhibited comparable physical and chemical stability. The following parameters (represented with * above) are recommended for evaluation in IVPT to assess impact on in vitro skin permeation: NA01 1% API, NA01 4% API, NA06 (10% NMP), NA07 (10% DMSO), NA11 (50% DMSO), NA09 (with Carbopol 974 and glycerol), and OG04 (Oleaginous gel).

### Example 4: Ointment Formulation Development

Tables 7A and 7B provide the ointment formulations tested for drug concentration using HPLC, stability utilizing the following tests: macroscopic appearance, microscopic appearance, physical stability and chemical stability at 25° C./65% RH and 40° C./75% RH, and viscosity using a Brookfield viscometer.

TABLE 7A
Set 1 of Ointment Formulations

| | PO05b 1% ACT | PO05b 4% ACT | PO09b ACT | PO10 | PO24 ACT | PO25b ACT |
|---|---|---|---|---|---|---|
| Component | Run 12 | Run 12 | Run 28 | SSPO16 | SSPO03 | SSPO22 |
| DMVT-505 | 1.00 | 4.00 | 1.00 | 1.00 | 1.00 | 1.00 |
| PEG 400 | 68.90 | 64.90 | 48.90 | 43.90 | 33.95 | 33.90 |
| Ethanol | 5.00 | 5.00 | | | | |
| Propylene grycol | | | | 15.00 | | |
| Dimethyl sulfoxide | | | | | | |
| (DMSO) | | | | | | |
| diethylene glycol | | | | 10.00 | | 40.00 |
| monoethyl ether | | | | | | |
| (DEGEE) | | | | | | |
| Water | | | | | 20.00 | |
| Glycerol | | | 25.00 | | 20.00 | |
| Dimethicone CST 100 | | | | | | |
| BHT | 0.10 | 0.10 | 0.10 | 0.10 | | 0.10 |
| Propyl gallate | | | | | 0.05 | |
| PEG 1500 | | | | | | |
| PEG 3350 | 25.00 | 25.00 | 25.00 | 30.00 | 25.00 | 25.00 |
| PEG 4000 | | | | | | |
| Total | 100.00 | 100.00 | 100.00 | 100.00 | 100.00 | 100.00 |

TABLE 7B
Set 2 of Ointment Formulations

| | PO29 ACT | PO32 | PO33 | PO35 | OO02 |
|---|---|---|---|---|---|
| Component | SSPO23 | N/A | N/A | N/A | N/A |
| DMVT-505 | 1.00 | 1.00 | 1.00 | 1.00 | 1.00 |
| PEG 400 | 53.90 | 68.90 | 68.90 | 13.90 | |
| Ethanol | — | 5.00 | 5.00 | — | |
| Propylene | — | — | — | — | |
| glycol | | | | | |
| Dimethyl | — | — | — | 50.00 | |
| sulfoxide | | | | | |
| (DMSO) | | | | | |
| diethylene | — | — | — | — | |
| glycol | | | | | |
| monoethyl | | | | | |
| ether | | | | | |
| (DEGEE) | | | | | |
| Water | — | — | — | — | |
| Glycerol | — | — | — | — | |
| Dimethicone | 10.00 | — | — | — | |
| CST 100 | | | | | |
| BHT | 0.10 | 0.10 | 0.10 | 0.10 | |
| Propyl gallate | — | — | — | — | |
| PEG 1500 | — | 25.00 | — | — | |
| PEG 3350 | 35.00 | — | — | 35.00 | |
| PEG 4000 | — | — | 25.00 | — | |
| Mineral Oil | | | | | 5.00 |
| White | | | | | 94.00 |
| petrolatum | | | | | |
| (white soft | | | | | |
| paraffin) | | | | | |
| Total | 100.00 | 100.00 | 100.00 | 100.00 | 100.00 |

Following formulation stability, all of the developed ointment formulations exhibited broadly comparable physical and chemical stability, with the exception of a very slight decrease in purity in PO35. The following parameters are recommended for evaluation in IVPT to assess impact on in vitro skin permeation: PO05b 1%, PO05b 4%, P010 (15% Propylene glycol and 10% diethylene glycol monoethyl ether (DEGEE)), PO25b (40% diethylene glycol monoethyl ether (DEGEE)), PO35 (50% DMSO), PO29 (with 35% PEG 3350), and OO02 (Hydrocarbon ointment).

### Example 5: Hybrid Formulation Development

Tables 8A, 8B, 8C, and 8D provide the hybrid formulations tested for drug concentration using HPLC, stability utilizing the following tests: macroscopic appearance, microscopic appearance, physical stability and chemical stability at 25° C./65% RH and 40° C./75% RH, and viscosity using a Brookfield viscometer.

TABLE 8A
Aqueous Emulsified Gels

| | EG01 | EG03 | EG04 |
|---|---|---|---|
| Component | ACT* | ACT* | ACT |
| DMVT-505 | 1.00 | 4.00 | 1.00 |
| PEG400 | — | 45.45 | 28.45 |
| Propylene glycol | — | 5.00 | 5.00 |
| Ethanol | — | 10.00 | 10.00 |
| diethylene glycol | 10.00 | 15.00 | 15.00 |
| monoethyl ether | | | |
| (DEGEE) | | | |
| Water | 62.90 | 10.00 | 30.00 |
| Phenoxyethanol | 1.00 | 1.00 | 1.00 |
| Glycerol | 5.00 | — | — |
| Acrylamide/Sodium | 4.00 | — | — |
| Acryloyldimethyl Taurate | | | |
| Copolymer/Isohexadecane | | | |
| & Polysorbate 80 (such as | | | |
| Sepineo P 600) | | | |
| triblock copolymer | — | 1.00 | 1.00 |
| consisting of a central | | | |
| hydrophobic block of | | | |
| polypropylene glycol | | | |
| flanked by two | | | |
| hydrophilic blocks of | | | |
| polyethylene glycol | | | |
| (PEG) (such as Poloxamer | | | |
| 407) | | | |
| crosslinked polyacrylic | — | 1.00 | 1.00 |
| acid (such as Carbomer | | | |
| Ultrez 10) | | | |
| BHT | 0.10 | — | — |
| Propyl gallate | — | 0.05 | 0.05 |
| Medium chain triglycerides | 10.00 | — | — |
| Dimethicone | 1.00 | — | — |
| Cyclomethicone | — | 7.50 | 7.50 |
| Isopropyl myristate | 5.00 | — | — |
| NaOH 0.2M | — | q.s. pH | q.s. pH |
| | | 6.0-6.5 | 6.0-6.5 |
| Water second addition | | q.s. 100% | q.s. 100% |
| Total | 100.00 | 100.00 | 100.00 |

TABLE 8B
Anhydrous Emulsified Gel

| | AEG04 ACT |
|---|---|
| Component | SSAEG03 |
| DMVT-505 | 1.00 |
| Glycerol | 30.50 |
| diethylene glycol | 20.00 |
| monoethyl ether | |
| (DEGEE) | |
| Propylene glycol | 31.50 |
| BHT | 0.05 |
| Hydroxyethyl | 1.50 |
| Acrylate/Sodium | |
| Acryloyldimethyl | |
| Taurate Copolymer | |
| (such as Sepineo | |
| D.E.R.M.) | |
| Cyclomethicone 5NF | 15.45 |
| Total | 100.00 |

TABLE 8C
Silicone-based Ointments

| | | SO04 | SO05 |
|---|---|---|---|
| Component | SO03ACT | ACT* | ACT |
| DMVT-505 | 1.00 | 1.00 | 1.00 |
| Petrolatum | 59.00 | 59.00 | 59.00 |
| Elastomer 10 | — | — | 20.00 |
| Dow Corning TI-3021 | 20.00 | 20.00 | — |
| Silicone Elastomer | | | |
| Blend | | | |
| Dimethicone 100 cps | 10.00 | — | — |
| Cyclomethicone 5NF | — | 10.00 | 10.00 |
| IPM | — | — | — |
| IPP | 10.00 | 10.00 | 10.00 |
| Total | 100.00 | 100.00 | 100.00 |

TABLE 8D
Hybrid Emulsions

| Component | ACR01ACT* | ACR02 ACT* | SCR04 ACT* | SCR05 ACT* |
|---|---|---|---|---|
| DMVT-505 | 1.00 | 1.00 | 1.00 | 1.00 |
| Isopropyl myristate | — | — | 10.00 | 10.00 |
| Dimethiconol Blend 20 | — | — | 4.00 | 4.00 |
| (mixture of | | | | |
| dimethicone and | | | | |
| dimethiconol) | | | | |
| TI-1050 fluid 350 Csp | — | — | 5.00 | 5.00 |
| (polydimethylsiloxane) | | | | |
| GILUGEL SIL 5 | — | — | 5.00 | — |
| (Cyclopentasiloxane (and) | | | | |
| Cyclohexasiloxane (and) | | | | |
| Aluminum/Magnesium | | | | |
| Hydroxide Stearate) | | | | |
| Span 83 (Sorbitan | — | — | 2.50 | 2.50 |
| Sesquioleate) | | | | |
| Hydroxyethyl | — | — | — | 1.50 |
| Acrylate/Sodium | | | | |
| Acryloyldimethyl | | | | |
| Taurate Copolymer | | | | |
| (such as Sepineo | | | | |
| D.E.R.M.) | | | | |
| Water | — | — | 55.50 | 60.00 |
| Glycerol | 5.00 | 5.00 | 15.00 | 15.00 |
| diethylene glycol | 20.00 | — | — | — |
| monoethyl ether | | | | |
| (DEGEE) | | | | |
| Propylene glycol | — | 20.00 | — | — |
| Phenoxyethanol | — | — | 1.00 | 1.00 |
| Sodium Chloride | — | — | 1.00 | — |
| Sedefos 75 (Mixture of | 20.00 | 20.00 | — | — |
| Triceteareth-4 | | | | |
| phosphate and | | | | |
| Ethylene glycol | | | | |
| palmitostearate and | | | | |
| Diethylene glycol | | | | |
| palmitostearate) | | | | |
| Labrafil M 2125 CS | 5.00 | 5.00 | — | — |
| (Linoleoyl polyoxyl-6 | | | | |
| glycerides NF/ | | | | |
| Linoleoyl macrogol-6 | | | | |
| glycerides EP) | | | | |
| Mineral oil | 10.00 | 10.00 | — | — |
| Compritol 888 | 5.00 | 5.00 | — | — |
| (Glyceryl Behenate) | | | | |
| Lauroglycol 90 | 35.00 | 35.00 | — | — |
| (Propylene glycol | | | | |
| monolaurate (type II) | | | | |
| EP/NF) | | | | |
| Total | 100.00 | 100.00 | 100.00 | 100.00 |

Following formulation stability, most of the developed ‘hybrid’ formulations exhibited broadly comparable physical and chemical stability, with the exception of variability in the drug recoveries and presence of crystals for the silicon-based ointments, and differences in the accelerated physical stability by LUMiSizer. The following parameters (represented by * above) are recommended for evaluation in IVPT to assess impact on in vitro skin permeation: EG03 (4% API), EG01 (10% diethylene glycol monoethyl ether (DEGEE) and 5% Isopropyl myristate), AEG04 ACT, ACR01 (20% diethylene glycol monoethyl ether (DEGEE)), ACR02 (20% Propylene glycol), SCR04 (5% Gilugel SIL 5 (Cyclopentasiloxane (and) Cyclohexasiloxane (and) Aluminum/Magnesium Hydroxide Stearate)), SCR05 (no Gilugel SIL 5), and 5004 (20% TI-3021 Silicone elastomer blend and 10% Cyclomethicone 5NF).

### Example 6: Foam Formulation Development

Tables 9A, 9B, and 9C provide the foam formulations tested for drug concentration using HPLC, stability utilizing the following tests: macroscopic appearance, microscopic appearance, physical stability and chemical stability at 25° C./65% RH and 40° C./75% RH, and viscosity using a Brookfield viscometer.

TABLE 9A
Set 1 of PEG-based Foams

| | FO01ACT* | FO04 ACT | FO17 ACT* | FO21 ACT* | FO22 ACT |
|---|---|---|---|---|---|
| Component | SSF1 | SSF1 | SSF11 | SSF8 | SSF9 |
| DMVT-505 | 1.00 | 1.00 | 1.00 | 1.00 | 1.00 |
| Water | 7.00 | 4.38 | — | — | — |
| Glycerol | — | — | 10.00 | 10.00 | 26.50 |
| diethylene glycol | 11.0 | 6.88 | 15.00 | 11.00 | 12.50 |
| monoethyl ether (DEGEE) | | | | | |
| PEG 400 | 38.50 | 23.69 | 29.00 | 29.88 | 21.50 |
| Propylene glycol | 7.40 | 4.63 | 10.00 | 6.62 | 8.00 |
| Benzyl alcohol | 1.50 | 0.94 | 1.50 | 1.50 | 1.50 |
| PEG 4000 | 12.60 | 7.88 | 7.50 | 9.00 | 8.00 |
| Brij S2 | 1.00 | 0.63 | 1.00 | 1.00 | 1.00 |
| Brij S20 | — | — | — | — | — |
| Aluminum starch | — | — | 5.00 | — | — |
| octenylsuccinate | | | | | |
| HFA134a | 20.00 | 50.00 | 20.00 | 30.00 | 20.00 |
| HFO1234ze | — | — | — | — | — |
| DME | — | — | — | — | — |
| Total | 100.00 | 100.00 | 100.00 | 100.00 | 100.00 |

TABLE 9B
Set 2 of PEG-based Foams

| | FO23 ACT | FO25 ACT* | FO30 ACT* | FO35 ACT |
|---|---|---|---|---|
| Component | SSF11 | SSF1 | SSF11 | SSF11 |
| DMVT-505 | 1.00 | 1.00 | 1.00 | 1.00 |
| Water | — | 7.00 | — | — |
| Glycerol | 8.50 | — | 8.50 | 8.50 |
| diethylene glycol | 15.00 | 11.00 | 15.00 | 15.00 |
| monoethyl ether | | | | |
| (DEGEE) | | | | |
| PEG 400 | 29.00 | 38.50 | 29.00 | 29.00 |
| Propylene glycol | 10.00 | 7.40 | 10.00 | 10.00 |
| Benzyl alcohol | 1.50 | 1.50 | 1.50 | 1.50 |
| PEG 4000 | 4.00 | 12.60 | 4.00 | 4.00 |
| Brij S2 | 1.00 | — | 1.00 | 1.00 |
| Brij S20 | — | 1.00 | — | — |
| Aluminum starch | 10.00 | — | 10.00 | 10.00 |
| octenylsuccinate | | | | |
| HFA134a | 20.00 | 20.00 | — | — |
| HFO1234ze | — | — | 20.00 | — |
| DME | — | — | — | 20.00 |
| Total | 100.00 | 100.00 | 100.00 | 100.00 |

TABLE 9C
Cream-based Foams

| | FO20 ACT* | FO27 ACT* |
|---|---|---|
| Component | SSF13 | CR01 |
| DMVT-505 | 1.00 | 1.00 |
| Water | 31.50 | 51.85 |
| Sodium citrate | — | 0.15 |
| dehydrate | | |
| Citric acid | — | 0.07 |
| monohydrate | | |
| EDTA | — | 0.08 |
| diethylene glycol | — | 1.62 |
| monoethyl ether | | |
| (DEGEE) | | |
| Propylene glycol | 15.00 | 8.08 |
| Polysorbate 80 | — | 1.21 |
| Benzyl alcohol | 1.00 | — |
| BHT | — | 0.08 |
| Benzoic Acid | — | 0.20 |
| Brij S20 | 3.00 | 0.53 |
| Brij S2 | — | 0.87 |
| Polawax NF | — | 3.50 |
| Glyceryl | 4.00 | — |
| monostearate | | |
| Cetosteartl alcohol | 1.50 | — |
| Mineral oil | 11.00 | — |
| White Soft Paraffin | 4.50 | — |
| Isopropyl Palmitate | 7.50 | — |
| Miglyol 810 | — | 10.75 |
| HFA134a | 20.00 | 20.00 |
| Total | 100.00 | 100.00 |

Following formulation stability, the drug chemical stability was higher in formulations with HFO or DME (FO30 and FO35) versus HFA (FO23) and with lower propellant levels (20% versus 30%). Levels of PEG 4000>9% in the formulations improved the physical stability. The following parameters (represented by * above) are recommended for evaluation in IVPT to assess impact on in vitro skin permeation: FO17 (7.5% PEG 4000), FO25 (12.6% PEG 4000), FO01 (20% HFA 134), FO30 (20% HFO), FO20 (15% Propylene glycol), FO27 (1.62% diethylene glycol monoethyl ether (DEGEE) and 8.08% Propylene glycol), FO21 (1% Brij S2), FO25 (1% Brij S20), and FO17 (5%).

### Example 7: Additional Foam Formulations Developed from a Cream

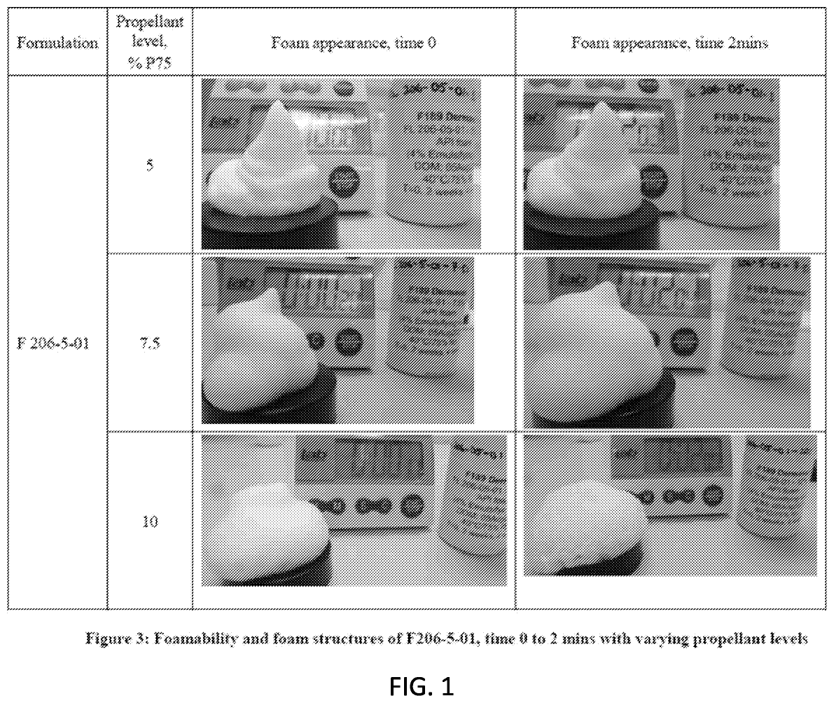

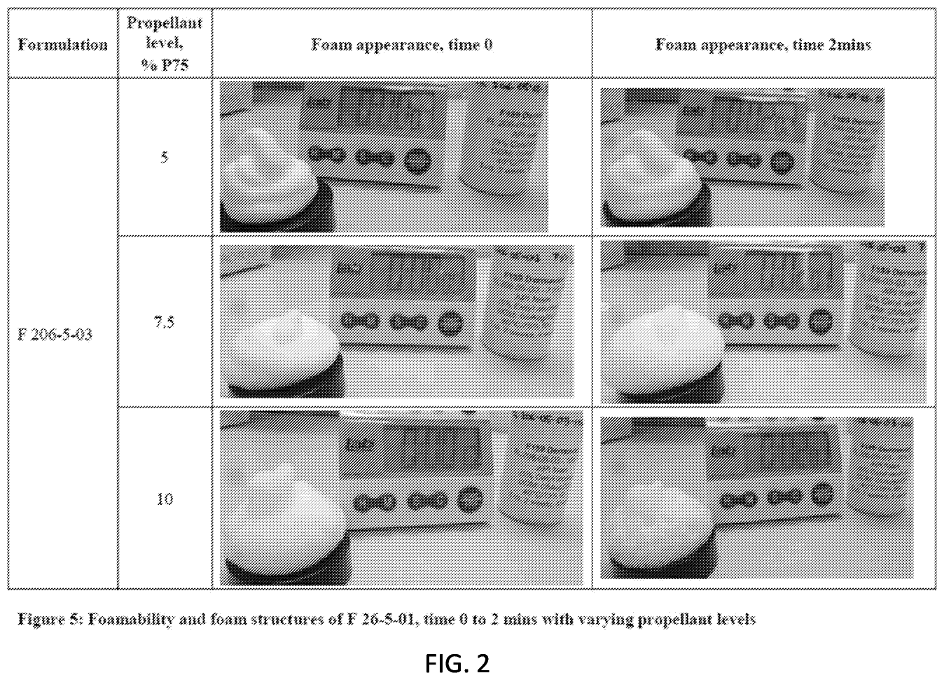

Development of foam formulations were focused on creating a stable foam. Table 10 provides 6 formulations developed from an effective cream formulation into foams using propane, butane and/or HFA-134A (1,1,1,2-tetrafluoroethane) as propellants. The tapinarof cream formulation described therein is converted to a foam formulation with the reduction of an emulsifying wax (Polawax), using 7.2% was not effective, 4% was ideal (formulations F1, F2, and F3) and 2% was suitable (formulations F4 and F6). FIG. 1 provides images of the foamability and foam structure of formulations F1, F2, F3, which have varying amounts of propellant, at 0 and 2 minutes. Replacing Polawax with 5% Cetyl alcohol resulted in an elegant foam on actuation (formulation F5). FIG. 2 provides images of the foamability and foam structure of formulation F5 with varying amounts of propellant at 0 and 2 minutes. All formulations phase separated during stability assessment at ambient and accelerated conditions; however, once shaken, formulations remained stable for at least 1 hr, which is considered sufficient for actuation.

TABLE 10

| | RLD 1% | | | | | | |
|---|---|---|---|---|---|---|---|
| | Tapinarof | | | | | | |
| | Cream | F1 | F2 | F3 | F4 | F5 | F6 |
| Ingredients | % w/w | % w/w | % w/w | % w/w | % w/w | % w/w | % w/w |
| Tapinarof (API) | 1.00 | 1.00 | 1.00 | 1.00 | 0.50 | 1.00 | 1.00 |
| Water | 64.67 | 67.87 | 68.37 | 67.87 | 69.87 | 66.87 | 69.87 |
| Citric Acid Anhydrous | 0.09 | 0.09 | 0.09 | 0.09 | 0.09 | 0.09 | 0.09 |
| Sodium Citrate Dihydrate | 0.19 | 0.19 | 0.19 | 0.19 | 0.19 | 0.19 | 0.19 |
| Disodium Edetate | 0.10 | 0.10 | 0.10 | 0.10 | 0.10 | 0.10 | 0.10 |
| Miglyol 812N (MCT) | 10.00 | 10.00 | 10.00 | 10.00 | 10.00 | 10.00 | 10.00 |
| Steareth-2 | 1.80 | 1.80 | 1.80 | 1.80 | 1.80 | 1.80 | 1.80 |
| Steareth-20 | 1.10 | 1.10 | 1.10 | 1.10 | 1.10 | 1.10 | 1.10 |
| Polysorbate 80 | 1.50 | 1.50 | 1.50 | 1.50 | 1.50 | 1.50 | 1.50 |
| Cetyl Alcohol | | | | | 5.00 | | |
| Emulsifying wax non-ionic | 7.20 | 4.00 | 4.00 | 4.00 | 2.00 | | 2.00 |
| (Polawax NF) | | | | | | | |
| BHT | 0.10 | 0.10 | 0.10 | 0.10 | 0.10 | 0.10 | 0.10 |
| Benzoic acid | 0.25 | 0.25 | 0.25 | 0.25 | 0.25 | 0.25 | 0.25 |
| Transcutol P | 2.00 | 2.00 | 2.00 | 2.00 | 2.00 | 2.00 | 2.00 |
| Propylene Glycol | 10.00 | 10.00 | 10.00 | 10.00 | 10.00 | 10.00 | 10.00 |
| TOTAL of Base | 100.00 | 100.00 | 100.00 | 100.00 | 100.00 | 100.00 | 100.00 |
| composition | | | | | | | |
| Propane | | 5% | 10% | | 5% | 10% | |
| Butane | | 5% | | | | | 7.5% |
| HFA-134A | | | | 10% | | | |

### Example 8: Novel Foam Formulations

Additional novel foam formulations were created with different emulsifying systems and thickeners in combination with a hydrocarbon and/or HFA-134A propellants. Table 11 provides 4 stable foam formulations.

TABLE 11

| | F7 | F8 | F9 | F10 |
|---|---|---|---|---|
| | (Foam 07 | (Foam 15 | (Foam 16 | (Modification of |
| | ACT) | ACT) | ACT) | Foam 07 ACT) |
| Ingredients | % w/w | % w/w | % w/w | % w/w |
| Tapinarof (API) | 1.00 | 1.00 | 1.00 | 1.00 |
| Water | 71.87 | 67.44 | 69.37 | 69.87 |
| Glycerin | 2.00 | 2.00 | 2.00 | 2.00 |
| Sodium Citrate | 0.19 | 0.19 | 0.19 | 0.19 |
| Dihydrate | | | | |
| Citric Acid | 0.09 | 0.09 | 0.09 | 0.09 |
| Anhydrous | | | | |
| Propylene glycol | 10.00 | 10.00 | 10.00 | 10.00 |
| Kolliwax CSA50 | 4.00 | 3.76 | 4.00 | 4.00 |
| Kolliphor CS20 | 5.50 | 5.17 | 5.50 | 5.50 |
| Medium Chain | 5.00 | 5.00 | 5.00 | 5.00 |
| Triglyceride | | | | |
| Cocoyl | — | — | 2.50 | — |
| Caprylocaprate | | | | |
| Isopropyl palmitate | — | 5.00 | — | — |
| Emulsifying wax | — | — | — | 2.00 |
| non-ionic | | | | |
| (Polawax NF) | | | | |
| BHT | 0.10 | 0.10 | 0.10 | 0.10 |
| Benzoic acid | 0.25 | 0.25 | 0.25 | 0.25 |
| TOTAL of Base | 100.00 | 100.00 | 100.00 | 100.00 |
| composition | | | | |
| Propane | 5% | 10% | | 10% |
| Butane | 5% | | 10% | |

1. An aqueous gel compositions comprising:
about 1% to about 4% tapinarof,
about 10% to about 65% water,
about 10% to about 50% diethylene glycol monoethyl ether (DEGEE),
about 5% to about 65% of a glycol,
about 2% to about 55% of a solvent,
about 0.5% to about 5% of a gelling agent, and
a neutralizing agent.

2. An anhydrous gel compositions comprising:
about 1% to about 4% tapinarof,
about 10% to about 70% of a solvent,
about 10% to about 30% diethylene glycol monoethyl ether (DEGEE),
about 15% to about 50% of a glycol, and
about 0.5% to about 5% of a gelling agent.

3. The composition of claim 1, wherein the composition has a pH of about 6 to about 6.5.

4. The composition of claim 1, wherein the solvent is selected from the group consisting of N-methyl-2-pyrrolidone (NMP), dimethyl sulfoxide (DMSO), dimethyl isosorbide (DMI), benzyl alcohol, phenoxyethanol, ethanol, cetostearyl alcohol, glycerol, medium chain triglycerides, D-panthenol, isostearic acid (such as Prisorine 3505), liquid medilan ultra, oleic acid, PEG 10 dimethicone, and combinations thereof.

5. The composition of claim 1, wherein the glycol is selected from the group consisting of low molecular weight PEG, PEG 200, PEG 300, PEG 400, propylene glycol, dipropylene glycol, hexylene glycol, butylene glycol, and combinations thereof.

6. The composition of claim 1, wherein the gelling agent is selected from the group consisting of a carbomer, an acrylate-based polymer, a cellulose-based polymer, a poloxamer, a high molecular weight PEG, a polyamide, a siligel, and combinations thereof.

7. The composition of claim 1, wherein the neutralizing agent is selected from the group consisting of sodium hydroxide, ammonium hydroxide, potassium hydroxide, arginine, aminomethyl propanol, tetrahydroxypropyl ethylenediamine, triethanolamine, tromethamine, PEG-15 cocamine, diisopropanolamine, triisopropanolamine, trolamine, and combinations thereof.

8. An ointment composition comprising:
about 1% to about 4% tapinarof,
about 10% to about 70% of a low molecular weight PEG,
about 50% to about 75% of a solvent, and
about 25% to about 35% of a high molecular weight PEG.

9. The ointment composition of claim 8, wherein the low molecular weight PEG is selected from the group consisting of PEG 200, PEG 300, PEG 400, PEG 500, PEG 600, and combinations thereof.

10. The ointment composition of claim 8, wherein the solvent is selected from the group consisting of a glycol, diethylene glycol monoethyl ether (DEGEE), an alcohol, water, glycerol, dimethyl sulfoxide (DMSO), N-methyl-2-pyrrolidone (NMP), dimethyl isosorbide (DMI), medium chain triglycerides, D-panthenol, isostearic acid (such as Prisorine 3505), liquid lanolin (such as liquid medilan ultra), oleic acid, PEG 10 dimethicone, and combinations thereof.

11. The ointment composition of claim 10, wherein the glycol is selected from the group consisting of propylene glycol, hexylene glycol, dipropylene glycol, butylene glycol, and combinations thereof.

12. The ointment composition of claim 10, wherein the alcohol is selected from the group consisting of ethanol, isopropanol, benzyl alcohol, phenoxyethanol, cetostearyl alcohol, and combinations thereof.

13. The ointment composition of claim 10, wherein the glycols are present in an amount of about 10% to about 40%.

14. The ointment composition of claim 10, wherein the diethylene glycol monoethyl ether (DEGEE) is present in an amount of about 10% to about 40%.

15. The ointment composition of claim 10, wherein the alcohol is present in an amount of about 1% to about 10%.

16. The ointment composition of claim 10, wherein the water is present in an amount of about 1% to about 25%.

17. The ointment composition of claim 10, wherein the glycerol is present in an amount of about 1% to about 25%.

18. The ointment composition of claim 10, wherein the dimethyl sulfoxide (DMSO) is present in an amount of about 1% to about 50%.

19. The ointment composition of claim 8, wherein the high molecular weight PEG is selected from the group consisting of PEG 1500, PEG 3350, PEG 4000, and combinations thereof.

20. The ointment composition of claim 8, wherein the ointment composition further comprises about 10% silicone-based solvent.

21. A foam composition comprising:
about 1% to about 4% tapinarof,
about 20% to about 80% of a solvent,
about 0.5% to about 10% of an emulsifier,
about 5% to about 15% of a thickener, and
about 20% to about 50% of a propellant.

22. The foam composition of claim 21, wherein the solvent is selected from the group consisting of a glycol, diethylene glycol monoethyl ether (DEGEE), an alcohol, water, glycerol, dimethyl sulfoxide (DMSO), N-methyl-2-pyrrolidone (NMP), dimethyl isosorbide (DMI), medium chain triglycerides, D-panthenol, isostearic acid (such as Prisorine 3505), liquid lanolin (such as liquid medilan ultra), oleic acid, PEG 10 dimethicone, and combinations thereof.

23. The foam composition of claim 22, wherein the glycol is selected from the group consisting of low molecular weight PEG, PEG 200, PEG 300, PEG 400, propylene glycol, dipropylene glycol, hexylene glycol, butylene glycol, and combinations thereof.

24. The foam composition of claim 22, wherein the alcohol is selected from the group consisting of ethanol, isopropanol, benzyl alcohol, phenoxyethanol, cetostearyl alcohol, and combinations thereof.

25. The foam composition of claim 21, wherein the emulsifiers are selected from the group consisting of steareth 2, steareth 20, polysorbate 80, polyoxyethylene (2) stearyl ether (such as Brij S2), polyoxyethylene (20) stearyl ether (such as Brij S20), Polawax NF, glyceryl monostearate, and combinations thereof.

26. The foam composition of claim 21, wherein the thickeners are selected from the group consisting of a carbomer, an acrylate-based polymer, a cellulose-based polymer, a poloxamer, a high molecular weight PEG, a polyamide, a siligel, aluminum starch octenylsuccinate, and combinations thereof.

27. The foam composition of claim 21, wherein the propellants are selected from the group consisting of hydro fluoro alkanes (HFA), hydro fluoro ethane (HFE), hydro fluoro olefine (HFO), propane, isobutane, butane, pentane, isopentane, dimethyl ether, and combinations thereof.

28. The foam composition of claim 21, further comprising an emollient selected from the group consisting of mineral oil, white soft paraffin, isopropyl palmitate, glycerin, propylene glycol, and combinations thereof.

29. A foam composition comprising a base composition of:
about 1% to about 2% tapinarof,
about 80% to about 90% of a solvent, and
about 8% to about 15% of an emulsifier, and
about 5% to about 10% of the total weight of the base composition a propellant.

30. A foam composition comprising a base composition of:
about 0.5% to about 2% tapinarof,
about 85% to about 95% of a solvent, and
about 3% to about 10% of an emulsifier, and
about 5% to about 10% of the total weight of the base composition a propellant.

31. A method of treating a dermatological condition or disorder in a patient in need thereof, the method comprising administering to said patient the topical pharmaceutical composition of claim 30.
